SEQUENCE LISTING (28 sequences)

SEQ ID NO: 1 - DNA, 1671 bp, Homo sapiens
  CDS (1)..(1671)
atgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgagggctgttatttcaagaaaccaagaagggccaggagaaatgggaaaagctgtgttgattcctaaagatgaccaggagaaaatgaaagagctgtttaaaatcaatcagtttaaccttatggccagtgatttgattgcccttaatagaagtctgccagatgtaagattagaaggatgtaagacaaaagtctaccctgatgaacttccaaacacaagtgtagtcattgtgtttcataatgaagcttggagcactctccttagaactgtttacagtgtgataaatcgttccccacactatctactctcagaggtcatcttggtagatgatgccagtgaaagagattttctcaagttgacattagagaattacgtgaaaaatttagaagtgccagtaaaaattattaggatggaagaacgctctgggttaatacgtgcccgtcttcgaggagcagctgcttcaaaagggcaggtcataacttttcttgatgcacactgtgaatgcacgttaggatggctggagcctttgctggcaagaataaaggaagacaggaaaacggttgtctgccctatcattgatgtgattagtgatgatacttttgaatatatggctgggtcagacatgacttatgggggttttaactggaaactgaatttccgctggtatcctgttccccaaagagaaatggacaggaggaaaggagacagaacattacctgtcaggacccctactatggctggtggcctattttctattgacagaaactactttgaagagataggaacttacgatgcaggaatggatatctggggtggagagaatcttgaaatgtcttttaggatttggcaatgtggaggctccttggagattgttacttgctcccatgttggtcatgtttttcggaaggcaactccatacacttttcctggtggcactggtcatgtcatcaacaagaacaacaggagactggcagaagtttggatggatgaatttaaagatttcttctacatcatatccccaggtgttgtcaaagtggattatggagatgtgtcagtcagaaaaacactaagagaaaatctgaagtgtaagcccttttcttggtacctagaaaacatctatccggactcccagatcccaagacgttattactcacttggtgagataagaaatgttgaaaccaatcagtgtttagacaacatgggccgcaaggaaaatgaaaaagtgggtatattcaactgtcatggtatgggaggaaatcaggtattttcttacactgctgacaaagaaatccgaaccgatgacttgtgcttggatgtttctagactcaatggacctgtaatcatgttaaaatgccaccatatgagaggaaatcagttatgggaatatgatgctgagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 2 - protein, 556 aa, Homo sapiens
MRRFVYCKVVLATSLMWVLVDVFLLLYFSECNKCDDKKERSLLPALRAVISRNQEGPGEMGKAVLIPKDDQEKMKELFKINQFNLMASDLIALNRSLPDVRLEGCKTKVYPDELPNTSVVIVFHNEAWSTLLRTVYSVINRSPHYLLSEVILVDDASERDFLKLTLENYVKNLEVPVKIIRMEERSGLIRARLRGAAASKGQVITFLDAHCECTLGWLEPLLARIKEDRKTVVCPIIDVISDDTFEYMAGSDMTYGGFNWKLNFRWYPVPQREMDRRKGDRTLPVRTPTMAGGLFSIDRNYFEEIGTYDAGMDIWGGENLEMSFRIWQCGGSLEIVTCSHVGHVFRKATPYTFPGGTGHVINKNNRRLAEVWMDEFKDFFYIISPGVVKVDYGDVSVRKTLRENLKCKPFSWYLENIYPDSQIPRRYYSLGEIRNVETNQCLDNMGRKENEKVGIFNCHGMGGNQVFSYTADKEIRTDDLCLDVSRLNGPVIMLKCHHMRGNQLWEYDAERLTLRHVNSNQCLDEPSEEDKMVPTMQDCSGSRSQQWLLRNMTLGT

SEQ ID NO: 3 - DNA, 1572 bp, Artificial Sequence
  GalNAc-T13DelEx9
atgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgagggctgttatttcaagaaaccaagaagggccaggagaaatgggaaaagctgtgttgattcctaaagatgaccaggagaaaatgaaagagctgtttaaaatcaatcagtttaaccttatggccagtgatttgattgcccttaatagaagtctgccagatgtaagattagaaggatgtaagacaaaagtctaccctgatgaacttccaaacacaagtgtagtcattgtgtttcataatgaagcttggagcactctccttagaactgtttacagtgtgataaatcgttccccacactatctactctcagaggtcatcttggtagatgatgccagtgaaagagattttctcaagttgacattagagaattacgtgaaaaatttagaagtgccagtaaaaattattaggatggaagaacgctctgggttaatacgtgcccgtcttcgaggagcagctgcttcaaaagggcaggtcataacttttcttgatgcacactgtgaatgcacgttaggatggctggagcctttgctggcaagaataaaggaagacaggaaaacggttgtctgccctatcattgatgtgattagtgatgatacttttgaatatatggctgggtcagacatgacttatgggggttttaactggaaactgaatttccgctggtatcctgttccccaaagagaaatggacaggaggaaaggagacagaacattacctgtcaggacccctactatggctggtggcctattttctattgacagaaactactttgaagagataggaacttacgatgcaggaatggatatctggggtggagagaatcttgaaatgtcttttaggatttggcaatgtggaggctccttggagattgttacttgctcccatgttggtcatgtttttcggaaggcaactccatacacttttcctggtggcactggtcatgtcatcaacaagaacaacaggagactggcagaagtttggatggatgaatttaaagatttcttctacatcatatccccaggtgttgtcaaagtggattatggagatgtgtcagtcagaaaaacactaagagaaaatctgaagtgtaagcccttttcttggtacctagaaaacatctatccggactcccagatcccaagacgttattactcacttggtgaggtattttcttacactgctgacaaagaaatccgaaccgatgacttgtgcttggatgtttctagactcaatggacctgtaatcatgttaaaatgccaccatatgagaggaaatcagttatgggaatatgatgctgagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 4 - protein, 523 aa, Artificial Sequence
  Synthetic Construct
MRRFVYCKVVLATSLMWVLVDVFLLLYFSECNKCDDKKERSLLPALRAVISRNQEGPGEMGKAVLIPKDDQEKMKELFKINQFNLMASDLIALNRSLPDVRLEGCKTKVYPDELPNTSVVIVFHNEAWSTLLRTVYSVINRSPHYLLSEVILVDDASERDFLKLTLENYVKNLEVPVKIIRMEERSGLIRARLRGAAASKGQVITFLDAHCECTLGWLEPLLARIKEDRKTVVCPIIDVISDDTFEYMAGSDMTYGGFNWKLNFRWYPVPQREMDRRKGDRTLPVRTPTMAGGLFSIDRNYFEEIGTYDAGMDIWGGENLEMSFRIWQCGGSLEIVTCSHVGHVFRKATPYTFPGGTGHVINKNNRRLAEVWMDEFKDFFYIISPGVVKVDYGDVSVRKTLRENLKCKPFSWYLENIYPDSQIPRRYYSLGEVFSYTADKEIRTDDLCLDVSRLNGPVIMLKCHHMRGNQLWEYDAERLTLRHVNSNQCLDEPSEEDKMVPTMQDCSGSRSQQWLLRNMTLGT

SEQ ID NO: 5 - DNA, 1632 bp, Artificial Sequence
  GalNAc-T13Del39bpEx9
atgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgagggctgttatttcaagaaaccaagaagggccaggagaaatgggaaaagctgtgttgattcctaaagatgaccaggagaaaatgaaagagctgtttaaaatcaatcagtttaaccttatggccagtgatttgattgcccttaatagaagtctgccagatgtaagattagaaggatgtaagacaaaagtctaccctgatgaacttccaaacacaagtgtagtcattgtgtttcataatgaagcttggagcactctccttagaactgtttacagtgtgataaatcgttccccacactatctactctcagaggtcatcttggtagatgatgccagtgaaagagattttctcaagttgacattagagaattacgtgaaaaatttagaagtgccagtaaaaattattaggatggaagaacgctctgggttaatacgtgcccgtcttcgaggagcagctgcttcaaaagggcaggtcataacttttcttgatgcacactgtgaatgcacgttaggatggctggagcctttgctggcaagaataaaggaagacaggaaaacggttgtctgccctatcattgatgtgattagtgatgatacttttgaatatatggctgggtcagacatgacttatgggggttttaactggaaactgaatttccgctggtatcctgttccccaaagagaaatggacaggaggaaaggagacagaacattacctgtcaggacccctactatggctggtggcctattttctattgacagaaactactttgaagagataggaacttacgatgcaggaatggatatctggggtggagagaatcttgaaatgtcttttaggatttggcaatgtggaggctccttggagattgttacttgctcccatgttggtcatgtttttcggaaggcaactccatacacttttcctggtggcactggtcatgtcatcaacaagaacaacaggagactggcagaagtttggatggatgaatttaaagatttcttctacatcatatccccaggtgttgtcaaagtggattatggagatgtgtcagtcagaaaaacactaagagaaaatctgaagtgtaagcccttttcttggtacctagaaaacatctatccggactcccagatcccaagacgttattactcacttggtgagataagaaatgttgaaaccaatcagtgtttagacaacatgggccgcaaggaaaatgaaaaagtattttcttacactgctgacaaagaaatccgaaccgatgacttgtgcttggatgtttctagactcaatggacctgtaatcatgttaaaatgccaccatatgagaggaaatcagttatgggaatatgatgctgagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 6 - protein, 543 aa, Artificial Sequence
  Synthetic Construct
MRRFVYCKVVLATSLMWVLVDVFLLLYFSECNKCDDKKERSLLPALRAVISRNQEGPGEMGKAVLIPKDDQEKMKELFKINQFNLMASDLIALNRSLPDVRLEGCKTKVYPDELPNTSVVIVFHNEAWSTLLRTVYSVINRSPHYLLSEVILVDDASERDFLKLTLENYVKNLEVPVKIIRMEERSGLIRARLRGAAASKGQVITFLDAHCECTLGWLEPLLARIKEDRKTVVCPIIDVISDDTFEYMAGSDMTYGGFNWKLNFRWYPVPQREMDRRKGDRTLPVRTPTMAGGLFSIDRNYFEEIGTYDAGMDIWGGENLEMSFRIWQCGGSLEIVTCSHVGHVFRKATPYTFPGGTGHVINKNNRRLAEVWMDEFKDFFYIISPGVVKVDYGDVSVRKTLRENLKCKPFSWYLENIYPDSQIPRRYYSLGEIRNVETNQCLDNMGRKENEKVFSYTADKEIRTDDLCLDVSRLNGPVIMLKCHHMRGNQLWEYDAERLTLRHVNSNQCLDEPSEEDKMVPTMQDCSGSRSQQWLLRNMTLGT

SEQ ID NO: 7 - DNA, 1869 bp, Artificial Sequence
  GalNAc-T13DelEx10B
atgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgagggctgttatttcaagaaaccaagaagggccaggagaaatgggaaaagctgtgttgattcctaaagatgaccaggagaaaatgaaagagctgtttaaaatcaatcagtttaaccttatggccagtgatttgattgcccttaatagaagtctgccagatgtaagattagaaggatgtaagacaaaagtctaccctgatgaacttccaaacacaagtgtagtcattgtgtttcataatgaagcttggagcactctccttagaactgtttacagtgtgataaatcgttccccacactatctactctcagaggtcatcttggtagatgatgccagtgaaagagattttctcaagttgacattagagaattacgtgaaaaatttagaagtgccagtaaaaattattaggatggaagaacgctctgggttaatacgtgcccgtcttcgaggagcagctgcttcaaaagggcaggtcataacttttcttgatgcacactgtgaatgcacgttaggatggctggagcctttgctggcaagaataaaggaagacaggaaaacggttgtctgccctatcattgatgtgattagtgatgatacttttgaatatatggctgggtcagacatgacttatgggggttttaactggaaactgaatttccgctggtatcctgttccccaaagagaaatggacaggaggaaaggagacagaacattacctgtcaggacccctactatggctggtggcctattttctattgacagaaactactttgaagagataggaacttacgatgcaggaatggatatctggggtggagagaatcttgaaatgtcttttaggatttggcaatgtggaggctccttggagattgttacttgctcccatgttggtcatgtttttcggaaggcaactccatacacttttcctggtggcactggtcatgtcatcaacaagaacaacaggagactggcagaagtttggatggatgaatttaaagatttcttctacatcatatccccaggtgttgtcaaagtggattatggagatgtgtcagtcagaaaaacactaagagaaaatctgaagtgtaagcccttttcttggtacctagaaaacatctatccggactcccagatcccaagacgttattactcacttggtgagataagaaatgttgaaaccaatcagtgtttagacaacatgggccgcaaggaaaatgaaaaagtgggtatattcaactgtcatggtatgggaggaaatcaggtattttcttacactgctgacaaagaaatccgaaccgatgacttgtgcttggatgtttctagactcaatggacctgtaatcatgttaaaatgccaccatatgagaggaaatcagttatgggaatatgatgctgagacccacactcttcttcatataatcacccagtcttgtctctcagtgaacaaagtagctgatggctcccagcatcctactgtggaaacctgtaatgatagcactttgcaaaaatggctactaagaaactatacaagaatggaaatttttagaaatatttttgggaattctactgattacattctctaatgatttttggagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 8 - protein, 571 aa, Artificial Sequence
  Synthetic Construct
MRRFVYCKVVLATSLMWVLVDVFLLLYFSECNKCDDKKERSLLPALRAVISRNQEGPGEMGKAVLIPKDDQEKMKELFKINQFNLMASDLIALNRSLPDVRLEGCKTKVYPDELPNTSVVIVFHNEAWSTLLRTVYSVINRSPHYLLSEVILVDDASERDFLKLTLENYVKNLEVPVKIIRMEERSGLIRARLRGAAASKGQVITFLDAHCECTLGWLEPLLARIKEDRKTVVCPIIDVISDDTFEYMAGSDMTYGGFNWKLNFRWYPVPQREMDRRKGDRTLPVRTPTMAGGLFSIDRNYFEEIGTYDAGMDIWGGENLEMSFRIWQCGGSLEIVTCSHVGHVFRKATPYTFPGGTGHVINKNNRRLAEVWMDEFKDFFYIISPGVVKVDYGDVSVRKTLRENLKCKPFSWYLENIYPDSQIPRRYYSLGEIRNVETNQCLDNMGRKENEKVGIFNCHGMGGNQVFSYTADKEIRTDDLCLDVSRLNGPVIMLKCHHMRGNQLWEYDAETHTLLHIITQSCLSVNKVADGSQHPTVETCNDSTLQKWLLRNYTRMEIFRNIFGNSTDYIL

SEQ ID NO: 9 - DNA, 657 bp, Artificial Sequence
  GalNAc-T13DelEx2-Ex7
atgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgaggggtgttgtcaaagtggattatggagatgtgtcagtcagaaaaacactaagagaaaatctgaagtgtaagcccttttcttggtacctagaaaacatctatccggactcccagatcccaagacgttattactcacttggtgagataagaaatgttgaaaccaatcagtgtttagacaacatgggccgcaaggaaaatgaaaaagtgggtatattcaactgtcatggtatgggaggaaatcaggtattttcttacactgctgacaaagaaatccgaaccgatgacttgtgcttggatgtttctagactcaatggacctgtaatcatgttaaaatgccaccatatgagaggaaatcagttatgggaatatgatgctgagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 10 - protein, 218 aa, Artificial Sequence
  Synthetic Construct
MRRFVYCKVVLATSLMWVLVDVFLLLYFSECNKCDDKKERSLLPALRGVVKVDYGDVSVRKTLRENLKCKPFSWYLENIYPDSQIPRRYYSLGEIRNVETNQCLDNMGRKENEKVGIFNCHGMGGNQVFSYTADKEIRTDDLCLDVSRLNGPVIMLKCHHMRGNQLWEYDAERLTLRHVNSNQCLDEPSEEDKMVPTMQDCSGSRSQQWLLRNMTLGT

SEQ ID NO: 11 - DNA, 1554 bp, Artificial Sequence
  GalNAc-T13DelEx6
atgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgagggctgttatttcaagaaaccaagaagggccaggagaaatgggaaaagctgtgttgattcctaaagatgaccaggagaaaatgaaagagctgtttaaaatcaatcagtttaaccttatggccagtgatttgattgcccttaatagaagtctgccagatgtaagattagaaggatgtaagacaaaagtctaccctgatgaacttccaaacacaagtgtagtcattgtgtttcataatgaagcttggagcactctccttagaactgtttacagtgtgataaatcgttccccacactatctactctcagaggtcatcttggtagatgatgccagtgaaagagattttctcaagttgacattagagaattacgtgaaaaatttagaagtgccagtaaaaattattaggatggaagaacgctctgggttaatacgtgcccgtcttcgaggagcagctgcttcaaaagggcaggtcataacttttcttgatgcacactgtgaatgcacgttaggatggctggagcctttgctggcaagaataaaggaagacaggaaaacggttgtctgccctatcattgatgtgattagtgatgatacttttgaatatatggctgggtcagacatgacttatgggggttttaactggaaactgaatttccgctggtatcctgttccccaaagagaaatggacaggaggaaaggagacagaacattacctgtcagatttggcaatgtggaggctccttggagattgttacttgctcccatgttggtcatgtttttcggaaggcaactccatacacttttcctggtggcactggtcatgtcatcaacaagaacaacaggagactggcagaagtttggatggatgaatttaaagatttcttctacatcatatccccaggtgttgtcaaagtggattatggagatgtgtcagtcagaaaaacactaagagaaaatctgaagtgtaagcccttttcttggtacctagaaaacatctatccgghactcccagatcccaagacgttattactcacttggtgagataagaaatgttgaaaccaatcagtgtttagacaacatgggccgcaaggaaaatgaaaaagtgggtatattcaactgtcatggtatgggaggaaatcaggtattttcttacactgctgacaaagaaatccgaaccgatgacttgtgcttggatgtttctagactcaatggacctgtaatcatgttaaaatgccaccatatgagaggaaatcagttatgggaatatgatgctgagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 12 - protein, 361 aa, Artificial Sequence
  Synthetic Construct
MRRFVYCKVVLATSLMWVLVDVFLLLYFSECNKCDDKKERSLLPALRAVISRNQEGPGEMGKAVLIPKDDQEKMKELFKINQFNLMASDLIALNRSLPDVRLEGCKTKVYPDELPNTSVVIVFHNEAWSTLLRTVYSVINRSPHYLLSEVILVDDASERDFLKLTLENYVKNLEVPVKIIRMEERSGLIRARLRGAAASKGQVITFLDAHCECTLGWLEPLLARIKEDRKTVVCPIIDVISDDTFEYMAGSDMTYGGFNWKLNFRWYPVPQREMDRRKGDRTLPVRFGNVEAPWRLLLAPMLVMFFGRQLHTLFLVALVMSSTRTTGDWQKFGWMNLKISSTSYPQVLSKWIMEMCQSEKH

SEQ ID NO: 13 - DNA, 1531 bp, Artificial Sequence
  GalNAc-T13DelEx8
atgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgagggctgttatttcaagaaaccaagaagggccaggagaaatgggaaaagctgtgttgattcctaaagatgaccaggagaaaatgaaagagctgtttaaaatcaatcagtttaaccttatggccagtgatttgattgcccttaatagaagtctgccagatgtaagattagaaggatgtaagacaaaagtctaccctgatgaacttccaaacacaagtgtagtcattgtgtttcataatgaagcttggagcactctccttagaactgtttacagtgtgataaatcgttccccacactatctactctcagaggtcatcttggtagatgatgccagtgaaagagattttctcaagttgacattagagaattacgtgaaaaatttagaagtgccagtaaaaattattaggatggaagaacgctctgggttaatacgtgcccgtcttcgaggagcagctgcttcaaaagggcaggtcataacttttcttgatgcacactgtgaatgcacgttaggatggctggagcctttgctggcaagaataaaggaagacaggaaaacggttgtctgccctatcattgatgtgattagtgatgatacttttgaatatatggctgggtcagacatgacttatgggggttttaactggaaactgaatttccgctggtatcctgttccccaaagagaaatggacaggaggaaaggagacagaacattacctgtcaggacccctactatggctggtggcctattttctattgacagaaactactttgaagagataggaacttacgatgcaggaatggatatctggggtggagagaatcttgaaatgtcttttaggatttggcaatgtggaggctccttggagattgttacttgctcccatgttggtcatgtttttcggaaggcaactccatacacttttcctggtggcactggtcatgtcatcaacaagaacaacaggagactggcagaagtttggatggatgaatttaaagatttcttctacatcatatccccagataagaaatgttgaaaccaatcagtgtttagacaacatgggccgcaaggaaaatgaaaaagtgggtatattcaactgtcatggtatgggaggaaatcaggtattttcttacactgctgacaaagaaatccgaaccgatgacttgtgcttggatgtttctagactcaatggacctgtaatcatgttaaaatgccaccatatgagaggaaatcagttatgggaatatgatgctgagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 14 - protein, 389 aa, Artificial Sequence [sequence omitted]

SEQ ID NO: 15 - DNA, 1413 bp, Artificial Sequence
  GalNAc-T13DelEx6DelEx8
atgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgagggctgttatttcaagaaaccaagaagggccaggagaaatgggaaaagctgtgttgattcctaaagatgaccaggagaaaatgaaagagctgtttaaaatcaatcagtttaaccttatggccagtgatttgattgcccttaatagaagtctgccagatgtaagattagaaggatgtaagacaaaagtctaccctgatgaacttccaaacacaagtgtagtcattgtgtttcataatgaagcttggagcactctccttagaactgtttacagtgtgataaatcgttccccacactatctactctcagaggtcatcttggtagatgatgccagtgaaagagattttctcaagttgacattagagaattacgtgaaaaatttagaagtgccagtaaaaattattaggatggaagaacgctctgggttaatacgtgcccgtcttcgaggagcagctgcttcaaaagggcaggtcataacttttcttgatgcacactgtgaatgcacgttaggatggctggagcctttgctggcaagaataaaggaagacaggaaaacggttgtctgccctatcattgatgtgattagtgatgatacttttgaatatatggctgggtcagacatgacttatgggggttttaactggaaactgaatttccgctggtatcctgttccccaaagagaaatggacaggaggaaaggagacagaacattacctgtcagatttggcaatgtggaggctccttggagattgttacttgctcccatgttggtcatgtttttcggaaggcaactccatacacttttcctggtggcactggtcatgtcatcaacaagaacaacaggagactggcagaagtttggatggatgaatttaaagatttcttctacatcatatccccagataagaaatgttgaaaccaatcagtgtttagacaacatgggccgcaaggaaaatgaaaaagtgggtatattcaactgtcatggtatgggaggaaatcaggtattttcttacactgctgacaaagaaatccgaaccgatgacttgtgcttggatgtttctagactcaatggacctgtaatcatgttaaaatgccaccatatgagaggaaatcagttatgggaatatgatgctgagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 16 - protein, 470 aa, Artificial Sequence
  Synthetic Construct
MRRFVYCKVVLATSLMWVLVDVFLLLYFSECNKCDDKKERSLLPALRAVISRNQEGPGEMGKAVLIPKDDQEKMKELFKINQFNLMASDLIALNRSLPDVRLEGCKTKVYPDELPNTSVVIVFHNEAWSTLLRTVYSVINRSPHYLLSEVILVDDASERDFLKLTLENYVKNLEVPVKIIRMEERSGLIRARLRGAAASKGQVITFLDAHCECTLGWLEPLLARIKEDRKTVVCPIIDVISDDTFEYMAGSDMTYGGFNWKLNFRWYPVPQREMDRRKGDRTLPVRFGNVEAPWRLLLAPMLVMFFGRQLHTLFLVALVMSSTRTTGDWQKFGWMNLKISSTSYPQIRNVETNQCLDNMGRKENEKVGIFNCHGMGGNQVFSYTADKEIRTDDLCLDVSRLNGPVIMLKCHHMRGNQLWEYDAERLTLRHVNSNQCLDEPSEEDKMVPTMQDCSGSRSQQWLLRNMTLGT

SEQ ID NO: 17 - DNA, 1572 bp, Artificial Sequence
  GalNAc-T13DelEx6DelEx8Del39bpEx9DelEx10B
atgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgagggctgttatttcaagaaaccaagaagggccaggagaaatgggaaaagctgtgttgattcctaaagatgaccaggagaaaatgaaagagctgtttaaaatcaatcagtttaaccttatggccagtgatttgattgcccttaatagaagtctgccagatgtaagattagaaggatgtaagacaaaagtctaccctgatgaacttccaaacacaagtgtagtcattgtgtttcataatgaagcttggagcactctccttagaactgtttacagtgtgataaatcgttccccacactatctactctcagaggtcatcttggtagatgatgccagtgaaagagattttctcaagttgacattagagaattacgtgaaaaatttagaagtgccagtaaaaattattaggatggaagaacgctctgggttaatacgtgcccgtcttcgaggagcagctgcttcaaaagggcaggtcataacttttcttgatgcacactgtgaatgcacgttaggatggctggagcctttgctggcaagaataaaggaagacaggaaaacggttgtctgccctatcattgatgtgattagtgatgatacttttgaatatatggctgggtcagacatgacttatgggggttttaactggaaactgaatttccgctggtatcctgttccccaaagagaaatggacaggaggaaaggagacagaacattacctgtcagatttggcaatgtggaggctccttggagattgttacttgctcccatgttggtcatgtttttcggaaggcaactccatacacttttcctggtggcactggtcatgtcatcaacaagaacaacaggagactggcagaagtttggatggatgaatttaaagatttcttctacatcatatccccagataagaaatgttgaaaccaatcagtgtttagacaacatgggccgcaaggaaaatgaaaaagtattttcttacactgctgacaaagaaatccgaaccgatgacttgtgcttggatgtttctagactcaatggacctgtaatcatgttaaaatgccaccatatgagaggaaatcagttatgggaatatgatgctgagacccacactcttcttcatataatcacccagtcttgtctctcagtgaacaaagtagctgatggctcccagcatcctactgtggaaacctgtaatgatagcactttgcaaaaatggctactaagaaactatacaagaatggaaatttttagaaatatttttgggaattctactgattacattctctaatgatttttggagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 18 - protein, 472 aa, Artificial Sequence
  Synthetic Construct
MRRFVYCKVVLATSLMWVLVDVFLLLYFSECNKCDDKKERSLLPALRAVISRNQEGPGEMGKAVLIPKDDQEKMKELFKINQFNLMASDLIALNRSLPDVRLEGCKTKVYPDELPNTSVVIVFHNEAWSTLLRTVYSVINRSPHYLLSEVILVDDASERDFLKLTLENYVKNLEVPVKIIRMEERSGLIRARLRGAAASKGQVITFLDAHCECTLGWLEPLLARIKEDRKTVVCPIIDVISDDTFEYMAGSDMTYGGFNWKLNFRWYPVPQREMDRRKGDRTLPVRFGNVEAPWRLLLAPMLVMFFGRQLHTLFLVALVMSSTRTTGDWQKFGWMNLKISSTSYPQIRNVETNQCLDNMGRKENEKVFSYTADKEIRTDDLCLDVSRLNGPVIMLKCHHMRGNQLWEYDAETHTLLHIITQSCLSVNKVADGSQHPTVETCNDSTLQKWLLRNYTRMEIFRNIFGNSTDYIL

SEQ ID NO: 19 - DNA, 18 bp, Artificial Sequence
  Primer
acatctatccggactccc

SEQ ID NO: 20 - DNA, 23 bp, Artificial Sequence
  Primer
tcatgtgcccaaggtcatgttcc

SEQ ID NO: 21 - protein, 60 aa, Artificial Sequence
  Synthetic Construct
MRKFAYCKVVLATSLIWVLLDMFLLLYFSECNKCDEKKERGLPAGDVLEPVQKPHEGPGE

SEQ ID NO: 22 - protein, 59 aa, Artificial Sequence
  Synthetic Construct
MRRSVYCKVVLATSLMWVLVDVFLLLYFSECNKCDDKKERSLLPALRAVISRNQEGPGE

SEQ ID NO: 23 - protein, 16 aa, Artificial Sequence
  Synthetic Construct
RSLLPALRAVISRNQE

SEQ ID NO: 24 - protein, 5 aa, Artificial Sequence
  Synthetic Construct
RSLLP

SEQ ID NO: 25 - protein, 6 aa, Artificial Sequence
  Synthetic Construct
LLPALR

SEQ ID NO: 26 - protein, 6 aa, Artificial Sequence
  Synthetic Construct
ALRAVI

SEQ ID NO: 27 - protein, 5 aa, Artificial Sequence
  Synthetic Construct
AVISR

SEQ ID NO: 28 - protein, 5 aa, Artificial Sequence
  Synthetic Construct
SRNQE

(19) United States
(12) Patent Application Publication
(10) Pub. No.: US 2016/0274115 A1
(43) Pub. Date: Sep. 22, 2016

(54) NOVEL METHOD TO DETECT RESISTANCE TO CHEMOTHERAPY IN PATIENTS WITH LUNG CANCER

(71) Applicant: Cedars-Sinai Medical Center, Los Angeles, CA (US)

(72) Inventors: Nora Berois, Montevideo (UY); Diego Touya, Lomas Solymar (UY); Mario Varangot, Montevideo (UY); Eduardo Osinaga, Montevideo (UY)

(73) Assignee: Cedars-Sinai Medical Center, Los Angeles, CA (US)

(21) Appl. No.: 15/152,367

(22) Filed: May 11, 2016

Related U.S. Application Data
(62) Division of application No. 14/416,608, filed on Jan. 22, 2015.
(60) Provisional application No. 61/675,276, filed on Jul. 24, 2012.

(51) Int. Cl. G01N 33/574; G01N 33/577

(52) U.S. Cl. CPC G01N 33/57423; G01N 33/577; G01N 2333/91142

(57) ABSTRACT

The invention is directed to processes, assays and methods for determining the likelihood of chemotherapy resistance and predicting response to chemotherapy in a subject with cancer. In an embodiment, the subject has lung cancer.

### FIELD OF INVENTION

The invention is directed to the role of an enzyme of the O-glycosylation pathway in the resistance of tumor cells to chemotherapy. Specifically, the invention provides a new molecular target, namely GalNAc-T13 (also known as ppGalNAc-T13), as a diagnostic marker of lung adenocarcinoma chemoresistance.

### BACKGROUND

Non-small cell lung cancer (NSCLC) continues to be the leading cause of cancer-related mortality in the United States and worldwide. NSCLC is classified by histology into adenocarcinoma, squamous cell carcinoma, and large cell carcinoma (Beasley et al., 2005). Adenocarcinoma has surpassed squamous cell histology in the United States as the most common type of NSCLC. Most cancer patients treated with chemotherapy will suffer severe toxicity, because response rates to a single therapy with anticancer drug are much lower than that to therapy for other diseases and also effective dose levels of anticancer drugs are often close to or overlap the toxic dose level. Thus, it is important to identify patients which are likely to be responsive to treatment with anticancer drugs. Development of biomarkers is necessary for predicting the effects of these agents on the relevant targets. The goal of the development of biomarkers is to design ways to predict efficacy of molecular-targeted agents including response rate, progression free survival (PFS) and overall survival (OS). If biomarkers allow us to select a patient population that might show a good treatment response, it would be beneficial to both patients and physicians (Saijo, 2012).

Glycoconjugates have proven to carry out relevant functions in cancer biology. Several diagnoses procedures based on detecting glycosylation alterations have been developed and incorporated to care practice (Adamczyk et al., 2012). O-gylcosylation alterations occur in most carcinomas, resulting in the expression of molecules which may constitute useful targets that can be exploited in diagnosis and prognosis (Reis et al., 2010), as well as for development of cancer vaccines (Tarp and Clausen, 2008). The synthesis of O-linked glycosylation is started in the Golgi apparatus by the covalent linkage of an α-N-acetylgalactosamine residue (GalNAc) to the hydroxyl group of Scr/Thr residues in a reaction catalyzed by UDP-N-acetyl-D-galactosamine:polypeptide N-acetylgalactosaminyltransferase (ppGalNAc-Ts, EC 2.4.2.41). ppGalNAc-Ts is a complex family of isoenzymes (Ten Hagen et al., 2003), of which 20 members have been characterized to date (Bennett et al., 2012). They have been found to be differentially expressed in malignant tissues compared to normal tissues (Mandel et al., 1999; Berois et al., 2006b). It was found that overexpression of GALNT3 gene promotes pancreatic cancer cell growth (Taniuchi et al., 2011) and that inactivating somatic and germline mutations of GALNT12 (a gene highly expressed in normal colon cells) are associated with colon cancer development (Guda et al., 2009). Increasing evidences suggest that these enzymes might be useful tumor markers. For example, it has been shown that GalNAc-T3 expression correlates with poor clinical outcome in patients with gallbladder cancer (Miyahara et al., 2004); GalNAc-T6 expression in bone marrow samples correlates with poor clinical outcome in lymph node-negative breast cancer patients (Freire et al., 2006). Regarding lung cancer, low expression of GalNAc-T3 may be a useful marker in predicting poor prognosis and early recurrence in patients with adenocarcinoma and with stage I diseases (Gu et al., 2004). The inventors have previously shown that GALNT13, the gene encoding the GalNAc-T13 isoenzyme, was the most up-regulated gene in metastatic neuroblasts compared with the primary tumor, and found that GALNT13 expression in bone marrow at diagnosis was a strong predictor of poor clinical outcome in neuroblastoma patients (Berois et al., 2006a). Here the inventors demonstrate that GalNAc-T13 is expressed in human lung cancer cells.

### SUMMARY OF THE INVENTION

The following embodiments and aspects thereof are described and illustrated in conjunction with systems, compositions and methods which are meant to be exemplary and illustrative, not limiting in scope.

Herein are described processes, assays and methods that include obtaining a sample comprising a tumor cell from a cancer patient desiring to know the likelihood of chemotherapy resistance, assaying the sample to determine the level of GalNac-T13 or a variant thereof and determining the subject has increased likelihood of chemotherapy resistance if the level of GalNac-T13 or a variant thereof is increased relative to a reference sample, or determining the subject has decreased likelihood of chemotherapy resistance if the level of GalNac-T13 or a variant thereof is the same as or decreased relative to the reference sample.

In various embodiments of the processes, assays and methods described herein, the subject is human. In some embodiments, the subject has undergone neoadjuvant therapy. In some embodiments, analyzing the level of GalNAc-T13 or a variant thereof in a sample obtained from the subject includes measuring the nucleic acid levels that encode GalNAc-T13 or a variant thereof, the protein levels of GalNAc-T13 or a variant thereof, or a combination thereof. In some embodiments, the sample from the subject is obtained before, during or after cancer treatment. In an embodiment, the subject has cancer, for example lung cancer. In an embodiment, lung cancer is non-small cell lung cancer (NSCLC). In a specific embodiment, the NSCLC is adenocarcinoma. In various embodiments, samples from the subject are obtained from tissue, blood, plasma or a combination thereof.

### BRIEF DESCRIPTION OF THE DRAWINGS

Exemplary embodiments are illustrated in the referenced figures. It is intended that the embodiments and figures disclosed herein are to be considered illustrative rather than restrictive

FIG. 1 depicts, in accordance with an embodiment of the invention, production and characterization of monoclonal antibodies specific for GalNAc-T13 (mAB T13.5).

FIG. 2 depicts, in accordance with an embodiment of the invention, GalNAc-T13 expression in human lung cancer cell lines. (A) RT-PCR for GALNT13: (1) Molecular weight marker (100 bp), (2) Negative control, (3) NCI-H1703 cell line, (4) NCI-H526 cell line, (5) NCI-H838 cell line, (6) SK-MES-1 cell line, (7) H69AR cell line, (8) H2O negative control, (9) NCI-H1755 cell line, (10) A549 cell line, (11) NCI-H1975 cell line, (12) NCI-H1650 cell line, (13) NL-20 cell line, (14) Positive control, BM cell line, (15) Molecular weight marker (100 bp). (B) Indirect immunofluorescence with mAb T13.5 in A549 lung cancer cell line. (C) Western blot with mAb T13.5: (1) Molecular weight marker, (2) BM cell line, (3) Hela cell line, (4) A549 cell line, (5) NCI-H1703 cell line.

FIG. 3 depicts, in accordance with an embodiment of the invention, a schematic representation of some splice variants of ppGalNAc-T13. We found 8 new transcripts generated by alternative splicing of ppGalNAc-T13. Sequences of the splice variants are set forth in SEQ ID NOs: 1-14.

FIG. 4 depicts, in accordance with an embodiment of the invention, immunohistochemistry in human lung cancer primary tumors with the monoclonal antibody T13.5.

FIG. 5 depicts, in accordance with an embodiment of the invention, (A) Kaplan-Meier survival estimates in patients with lung adenocarcinoma which received neoadjuvant therapy with GalNAc-T13 expression in primary tumors; (B) Kaplan-Meier survival estimates in patients with advanced lung adenocarcinoma which received neoadjuvant therapy with GalNAc-T13 expression in primary tumors; (C) Kaplan-Meier survival estimates in patients with early stage lung adenocarcinoma which received neoadjuvant therapy with GalNAc-T13 expression in primary tumors.

### DETAILED DESCRIPTION OF THE INVENTION

All references cited herein are incorporated by reference in their entirety as though fully set forth.

Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. In some embodiments, the disease condition is cancer.

“Subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented. Examples of cancer treatment include, but are not limited to, active surveillance, observation, surgical intervention, chemotherapy, immunotherapy, radiation therapy (such as external beam radiation, stereotactic radiosurgery (gamma knife), and fractionated stereotactic radiotherapy (FSR)), focal therapy, systemic therapy, vaccine therapies, viral therapies, molecular targeted therapies, or a combination thereof.

“Tumor,” as used herein refers to all neoplastic cell growth and proliferation, whether malignant or benign, and all pre-cancerous and cancerous cells and tissues.

“Cancer” and “cancerous” refer to or describe the physiological condition in mammals that is typically characterized by unregulated cell growth. Examples of cancer include, but are not limited to B-cell lymphomas (Hodgkin's lymphomas and/or non-Hodgkins lymphomas), brain cancer, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer, including but not limited to androgen-dependent prostate cancer and androgen-independent prostate cancer.

“Chemotherapy resistance” as used herein refers to partial or complete resistance to chemotherapy drugs. For example, a subject does not respond or only partially responds to a chemotherapy drug. A person of skill in the art can determine whether a subject is exhibiting resistance to chemotherapy.

“Chemotherapeutic drugs” or “chemotherapeutic agents” as used herein refer to drugs used to treat cancer including but not limited to Albumin-bound paclitaxel (nab-paclitaxel), Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, or a combination thereof.

“Patient outcome” refers to whether a patient survives or dies as a result of treatment. A more accurate prognosis for patients as provided in this invention increases the chances of patient survival.

“Poor Prognosis” means that the prospect of survival and recovery of disease is unlikely despite the standard of care for the treatment of the cancer (for example, lung cancer), that is, surgery, radiation, chemotherapy. Poor prognosis is the category of patients whose survival is less than that of the median survival.

“Good Prognosis” means that the prospect of survival and recovery of disease is likely with the standard of care for the treatment of the disease, for example, surgery, radiation, chemotherapy. Good prognosis is the category of patients whose survival is not less than that of the median survival.

A “recurrence” means that the cancer has returned after initial treatment.

“Variant” as used herein refers to a mutant GalNAc-T13, a splice variant of GalNAc-T13 or a combination thereof. A mutant of GalNAc-T13 may be a result of an insertion, deletion, missense, nonsense and/or a truncation mutation in the gene encoding GalNAc-T13.

Being “non-recurrent” or “recurrence-free” means that the cancer is in remission; being recurrent means that the cancer is growing and/or has metastasized, and some surgery, therapeutic intervention, and/or cancer treatment is required to lower the chance of lethality. The “non-recurrent subjects” are subjects who have non-recurrent or recurrence-free disease, and they can be used as the control for recurrent subjects who have recurrent disease or recurrence

O-gylcosylation alterations occur in most carcinomas, resulting in the expression of molecules which may constitute useful targets for diagnosis and therapy. GalNAc-T13 enzyme catalyzes a key step in the initiation of O-glycosylation. It is overexpressed in metastatic neuroblastoma and has been correlated with the prognosis of patients with this tumor. In resected lung cancer specimens there is no information about GalNAc-T13 expression.

As described herein, Applicants observed increased GalNAc-T13 expression in NSCLC, without significant differences between subjects with neoadjuvant (WNA) chemotherapy and without neoadjuvant (WONA) chemotherapy. GalNAc-T13 is expressed in NSCLC and associates with poor prognosis in patients with adenocarcinomas (ADCA) who received neoadjuvant chemotherapy. Applicants' data suggested that GalNAc-T13 is a novel marker associated to chemoresistance in NSCLC.

Accordingly, the invention is based, at least in part, on these findings. The present invention addresses the need for molecular indicators for the prognostication of cancer, such as lung cancer, for determination of chemotherapy resistance in cancer patients and for guiding treatment options in cancer patients. The invention provides processes, assays and methods for determining the likelihood of chemotherapy resistance in cancer patients so as to optimize cancer therapy in a subject in need thereof.

Specifically, the invention provides a process comprising obtaining a sample comprising a cancer cell from a cancer patient desiring to know the likelihood of chemotherapy resistance, analyzing the sample to determine the level of GalNac-T13 or a variant thereof and determining the subject has increased likelihood of chemotherapy resistance if the level of GalNac-T13 or a variant thereof is increased relative to a reference sample, or determining the subject has decreased likelihood of chemotherapy resistance if the level of GalNac-T13 or a variant thereof is the same as of decreased relative to the reference sample. In an embodiment, the subject has lung cancer.

In some embodiments, the process may further comprise prescribing a first therapy to the subject if the subject has decreased likelihood of chemotherapy resistance or prescribing a second therapy to the subject if the subject has increased likelihood of chemotherapy resistance. In some embodiments, the first therapy may be any one or more of surgery, radiation, chemotherapy, immunotherapy, vaccine, or a combination thereof. In some embodiments, the second therapy may be non-chemotherapy comprising therapy and many be any one or more of surgery, radiation, immunotherapy, vaccine, or a combination thereof. In additional embodiments, the second therapy may be any one or more of surgery, radiation, chemotherapy, immunotherapy, vaccine, or a combination thereof, wherein chemotherapy includes administering to the subject one or more chemotherapeutic agents that have not been used previously to treat the subject or administering a chemotherapeutic agent that has been previously administered to the subject but at a dose higher than previously administered.

In some embodiments, the second therapy may include selecting non-chemotherapy-comprising cancer therapy for the subject when the expression of GalNAc-T13 or a variant thereof in the sample from the subject is increased compared to the reference sample based on the recognition that chemotherapy may not be effective in subject whose cancer has increased expression of GalNAc-T13 or a variant thereof. In further embodiments, the second therapy may include selecting chemotherapy-comprising cancer therapy when the expression of GAalNAc-T13 or a variant thereof in the sample from the subject is the same as or decreased compared to the reference sample based on the recognition that chemotherapy may be effective in the subject whose cancer has decreased expression of GalNAc-T13 or a variant thereof.

The invention also provides an assay comprising obtaining a sample comprising a cancer cell from a cancer patient desiring to know the likelihood of chemotherapy resistance, analyzing the sample to determine the level of GalNac-T13 or a variant thereof and determining the subject has increased likelihood of chemotherapy resistance if the level of GalNac-T13 or a variant thereof is increased relative to a reference sample, or determining the subject has decreased likelihood of chemotherapy resistance if the level of GalNac-T13 or a variant thereof is the same as of decreased relative to the reference sample. In an embodiment, the subject has lung cancer.

The invention further provides an assay for determining the likelihood of chemotherapy resistance in a subject in need thereof. The assay includes providing a biological sample from a subject having cancer, providing an antibody that specifically binds to GalNAc-T13 or a variant thereof, contacting the biological sample with the antibody and detecting (for example using immunoassay) the level of antibody binding to GalNAc-T13 or a variant thereof, wherein an increase in binding in the biological sample from the subject relative to a reference sample is indicative of increased likelihood of chemotherapy resistance in the subject. In an embodiment, the cancer is lung cancer. In an embodiment, the antibody is the T13.5 antibody described herein that binds an epitope having the sequence LLPALR in GalNAc-T13 or a variant thereof.

In some embodiments, assay for determining the likelihood of chemotherapy resistance in a subject may include providing a biological sample from a subject having cancer and determining the level of mRNA present in a sample obtained from the subject that encodes GalNAc-T13 or a variant thereof. An increase in the mRNA level in the sample obtained from the subject relative to the reference sample is indicative of increased likelihood of chemotherapy resistance in the subject. In an embodiment, the cancer is lung cancer.

The assays of the invention may further comprise selecting and/or administering a therapy to treat, reduce, inhibit or reduce the severity of cancer in the subject. Selecting the therapy includes prescribing a first therapy to the subject if the subject has decreased likelihood of chemotherapy resistance or prescribing a second therapy to the subject if the subject has increased likelihood of chemotherapy resistance. In some embodiments, the first therapy is any one or more of surgery, radiation, chemotherapy, immunotherapy, vaccine, or a combination thereof. In some embodiments, the second therapy may be non-chemotherapy comprising therapy and may be any one or more of surgery, radiation, immunotherapy, vaccine, or a combination thereof. In additional embodiments, the second therapy is any one or more of surgery, radiation, chemotherapy, immunotherapy, vaccine, or a combination thereof, wherein chemotherapy comprises administering to the subject one or more chemotherapeutic agents that have not been used previously to treat the subject or administering a chemotherapeutic agent that has been previously administered to the subject but at a dose higher than previously administered. In some embodiments, the cancer is lung cancer.

In some embodiments, the second therapy may include selecting non-chemotherapy comprising cancer therapy for treatment of cancer in the subject. In some embodiments, the assay further comprises selecting non-chemotherapy-comprising cancer therapy for the subject when the expression of GalNAc-T13 or a variant thereof in the sample from the subject is increased compared to the reference sample based on the recognition that chemotherapy may not be effective in a subject whose cancer has increased expression of GalNAc-T13 or a variant thereof. In further embodiments, the second therapy may include selecting chemotherapy-comprising cancer therapy when the expression of GalNAc-T13 or a variant thereof in the sample from the subject is the same as or decreased compared to the reference sample based on the recognition that chemotherapy may be effective in the subject whose cancer has decreased expression of GalNAc-T13 or a variant thereof.

The invention also provides methods comprising obtaining a sample comprising a cancer cell from a cancer patient desiring to know the likelihood of chemotherapy resistance, analyzing the sample to determine the level of GalNAc-T13 or a variant thereof and determining the subject has increased likelihood of chemotherapy resistance if the level of GalNAc-T13 or a variant thereof is increased relative to a reference sample, or determining the subject has decreased likelihood of chemotherapy resistance if the level of GalNAc-T13 or a variant thereof is the same as of decreased relative to the reference sample. In an embodiment, the subject has lung cancer.

The invention further provides a method for selecting treatment for a subject having cancer, and optionally administering the treatment/therapy comprising providing a biological sample from a subject having cancer, providing an antibody that specifically binds to GalNAc-T13, contacting the biological sample with the antibody, detecting (for example, using immunoassays) whether the antibody binds GalNAc-T13 and selecting a therapy. The method further comprises administering the selected therapy. In an embodiment of the method, the presence of binding of the antibody to GalNAc-T13 in the biological sample from the subject relative to a reference sample is indicative of increased expression of GalNAc-T13 and increased likelihood of chemotherapy resistance in the subject. In an embodiment, the cancer is lung cancer. In an embodiment, the antibody is the T13.5 antibody described herein that binds an epitope having the sequence LLPALR in GalNAc-T13 or a variant thereof.

The invention also provides a method for selecting treatment for a subject having cancer, and optionally administering the treatment/therapy comprising providing a biological sample from a subject having cancer and determining the level of mRNA present in a sample obtained from the subject that encodes GalNAc-T13 or a variant thereof. An increase in the mRNA level in the sample obtained from the subject relative to the reference sample is indicative of increased likelihood of chemotherapy resistance in the subject. In an embodiment, the cancer is lung cancer.

In some embodiments, selecting a therapy includes prescribing a first therapy to the subject if the subject has decreased likelihood of chemotherapy resistance or prescribing a second therapy to the subject if the subject has increased likelihood of chemotherapy resistance. In some embodiments, the first therapy is any one or more of surgery, radiation, chemotherapy, immunotherapy, vaccine, or a combination thereof. In some embodiments, the second therapy may be non-chemotherapy comprising therapy and may be any one or more of surgery, radiation, immunotherapy, vaccine, or a combination thereof. In additional embodiments, the second therapy is any one or more of surgery, radiation, chemotherapy, immunotherapy, vaccine, or a combination thereof, wherein chemotherapy comprises administering to the subject one or more chemotherapeutic agents that have not been used previously to treat the subject or administering a chemotherapeutic agent previously administered to the subject at a dose higher than previously administered.

The invention further provides an isolated sample obtained from a human subject comprising an abnormal level of GalNAc-T13. In some embodiments, the sample is any one or more of tissue, blood, plasma, urine or a combination thereof.

The invention also provides combinations of an isolated sample obtained from a human subject that includes an abnormal level of GalNAc-T13 and a reagent which reacts with the GalNAc-T13. In an embodiment, the reagent comprises a label to produce a signal indicative of the presence of the abnormal level of the GalNAc-T13 in the isolated sample. In some embodiments, the label is any one or more of a radiolabel, a chromophore, a fluorophore or a combination thereof. In various embodiments, the reagent is any one or more of a GalNAc-T13-specific nucleic acid, a ppGalNAc-T13-specific monoclonal antibody, a GalNAc-T13-enyme-specific substrate, a small molecule, a lipid or a combination thereof.

The invention also provides a system that includes an isolated sample obtained from a human subject, comprising an abnormal level of GalNAc-T13 and a reagent to react with the GalNAc-T13. In an embodiment, the reagent comprises a label to produce a signal indicative of the presence of the abnormal level of the GalNAc-T13 in the isolated sample. In some embodiments, the label is any one or more of a radiolabel, a chromophore, a fluorophore or a combination thereof. In various embodiments, the reagent is any one or more of a GalNAc-T13-specific nucleic acid, a GalNAc-T13-specific monoclonal antibody, a GalNAc-T13-enyme-specific substrate, a small molecule, a lipid or a combination thereof.

In various embodiments of the processes, assays and methods described herein, the subject is human. In some embodiments, the subject has undergone neoadjuvant therapy (for example, neoadjuvant therapy using any one or more of carboplatin, paclitaxel, carboplatin, cisplatin, docetaxel, gemcitabine, etoposido, pemetrexed, cetuximab, or a combination thereof). In some embodiments, analyzing the level of GalNAc-T13 or a variant thereof in a sample obtained from the subject includes measuring the nucleic acid levels that encode GalNAc-T13 or a variant thereof, the protein levels of GalNAc-T13 or a variant thereof, or a combination thereof. In some embodiments, the sample from the subject is obtained before, during or after cancer treatment. In an embodiment, the subject has cancer, for example lung cancer. In an embodiment, lung cancer is non-small cell lung cancer (NSCLC). In a specific embodiment, the NSCLC is adenocarcinoma. In various embodiments, samples from the subject are obtained from tissue, blood, plasma or a combination thereof.

### Analysis of GalNAc-T13 Expression

In various embodiments of the processes, assays and methods described herein, assaying the GalNAc-T13 or a variant thereof comprises measuring the amount of nucleic acid encoding GalNAc-T13 or a variant thereof present in the sample, measuring the amount of GalNAc-T13 protein or a variant thereof protein present in the sample, or a combination thereof.

In various embodiments of the processes, assays and methods described herein, analyzing the sample includes detecting the level of GalNAc-T13 or a variant thereof with an antibody specific to GalNAc-T13 or a variant thereof. In various embodiments, the antibody is any one or more of a monoclonal antibody or fragment thereof, a polyclonal antibody or a fragment thereof, chimeric antibodies, humanized antibodies, human antibodies, and a single chain antibody. In an embodiment, the antibody is a monoclonal antibody. An example of a monoclonal antibody that may be used is the T13.5 monoclonal antibody that binds the sequence LLPALR of GalNAc-T13 or a variant thereof.

In some embodiments of the processes, assays and methods described herein, analyzing the sample includes measuring the levels mRNA that encode GalNAc-T13 or a variant thereof, present in the sample with a polynucleotide capable of hybridizing with mRNA specific for GalNAc-T13 or a variant thereof under stringent hybridization conditions.

Techniques that may be used to assess the amount of nucleic acid encoding ppGalNAc-T13 or a variant thereof, present in the sample include but are not limited to in situ hybridization (e.g., Angerer (1987) Meth. Enzymol 152: 649). Preferred hybridization-based assays include, but are not limited to, traditional “direct probe” methods such as Southern blots or in situ hybridization (e.g., FISH and FISH plus SKY), and “comparative probe” methods such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches. Probes that may be used for nucleic acid analysis are typically labeled, e.g., with radioisotopes or fluorescent reporters. Preferred probes are sufficiently long so as to specifically hybridize with the target nucleic acid(s) under stringent conditions. The preferred size range is from about 200 bases to about 1000 bases. Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), Pinkel, et al. (1998) Nature Genetics 20: 207-211, and/or Kallioniemi (1992) Proc. Natl Acad Sci USA 89:5321-5325 (1992).

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and sybr green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

A two-tailed student t-test with unequal variation may be used to measure the differences between the patient's expression of GalNAc-T13 and a normal blood sample, or the patient's own blood (matched control), or a reference generate by computer algorithm pooling many control samples, as described herein. A significant difference may be achieved where the p value is equal to or less than 0.05. GalNAc-T13 mRNA expression may also be used to determine patient's prognosis and response to chemotherapy, where GalNAc-T13 mRNA expression is separated into two groups: those with high ppGalNAc-T13 expression and those with low or no detectable GalNAc-T13 expression. The groups may be separated by the median GalNAc-T13 expression and plotted over time with a Kaplan-Meier curve.

Suitable methods for assaying the expression level of GalNAc-T13 or a variant thereof include but are not limited to using DNA sequencing, comparative genomic hybridization (CGH), array CGH (aCGH), SNP analysis, mRNA expression assay, RT-PCR, real-time PCR, or a combination thereof. In various embodiments, the assay to detect the nucleic acid encoding or protein levels of, GalNAc-T13, is any one or more of Northern blot analysis, Southern blot analysis, reverse transcription-polymerase chain reaction (RT-PCR), polymerase chain reaction (PCR), enzyme-linked immunosorbent assay (ELISA), radio-immuno assay (RIA), sodium dodecyl sulfate polyacrylamide gel electrophoresis (SDS-PAGE), Western blot analysis or a combination thereof. In some embodiments, the level of GalNAc-T13 in a subject may be ascertained by measuring the substrate upon which the enzyme GalNAc-T13 acts, such that the substrate serves as a surrogate marker for GalNAc-T13.

Antibodies, both polyclonal and monoclonal, can be produced by a skilled artisan either by themselves using well known methods or they can be manufactured by service providers who specialize making antibodies based on known protein sequences. In the present invention, the protein sequences are known and thus production of antibodies against them is a matter of routine.

For example, production of monoclonal antibodies can be performed using the traditional hybridoma method by first immunizing mice with an antigen which may be an isolated protein of choice or fragment thereof (for example, GalNAc-T13 or a fragment thereof or a variant thereof) and making hybridoma cell lines that each produce a specific monoclonal antibody. The antibodies secreted by the different clones are then assayed for their ability to bind to the antigen using, e.g., ELISA or Antigen Microarray Assay, or immuno-dot blot techniques. The antibodies that are most specific for the detection of the protein of interest can be selected using routine methods and using the antigen used for immunization and other antigens as controls. The antibody that most specifically detects the desired antigen and protein and no other antigens or proteins are selected for the processes, assays and methods described herein.

The best clones can then be grown indefinitely in a suitable cell culture medium. They can also be injected into mice (in the peritoneal cavity, surrounding the gut) where they produce an antibody-rich ascites fluid from which the antibodies can be isolated and purified. The antibodies can be purified using techniques that are well known to one of ordinary skill in the art.

In the methods and assays of the invention, the presence of any GalNAc-T13 or a fragment thereof is determined using antibodies specific for the GalNAc-T13 protein or a fragment or variant thereof and detecting immunospecific binding of each antibody to its respective cognate marker.

Any suitable immunoassay method may be utilized, including those which are commercially available, to determine the level GalNAc-T13 or a variant thereof measured according to the invention. Extensive discussion of the known immunoassay techniques is not required here since these are known to those of skill in the art. Typical suitable immunoassay techniques include sandwich enzyme-linked immunoassays (ELISA), radioimmunoassays (RIA), competitive binding assays, homogeneous assays, heterogeneous assays, etc. Various known immunoassay methods are reviewed, e.g., in Methods in Enzymology, 70, pp. 30-70 and 166-198 (1980).

In the assays of the invention, “sandwich-type” assay formats can be used. Some examples of such sandwich-type assays are described in by U.S. Pat. No. 4,168,146 to Grubb, et al. and U.S. Pat. No. 4,366,241 to Tom, et al. An alternative technique is the “competitive-type” assay. In a competitive assay, the labeled probe is generally conjugated with a molecule that is identical to, or an analog of, the analyte. Thus, the labeled probe competes with the analyte of interest for the available receptive material. Competitive assays are typically used for detection of analytes such as haptens, each hapten being monovalent and capable of binding only one antibody molecule. Examples of competitive immunoassay devices are described in U.S. Pat. No. 4,235,601 to Deutsch, et al., U.S. Pat. No. 4,442,204 to Liotta, and U.S. Pat. No. 5,208,535 to Buechler, et al.

The antibodies can be labeled. In some embodiments, the detection antibody is labeled by covalently linking to an enzyme, label with a fluorescent compound or metal, label with a chemiluminescent compound. For example, the detection antibody can be labeled with catalase and the conversion uses a colorimetric substrate composition comprises potassium iodide, hydrogen peroxide and sodium thiosulphate; the enzyme can be alcohol dehydrogenase and the conversion uses a colorimetric substrate composition comprises an alcohol, a pH indicator and a pH buffer, wherein the pH indicator is neutral red and the pH buffer is glycine-sodium hydroxide; the enzyme can also be hypoxanthine oxidase and the conversion uses a colorimetric substrate composition comprises xanthine, a tetrazolium salt and 4,5-dihydroxy-1,3-benzene disulphonic acid. In one embodiment, the detection antibody is labeled by covalently linking to an enzyme, label with a fluorescent compound or metal, or label with a chemiluminescent compound.

Direct and indirect labels can be used in immunoassays. A direct label can be defined as an entity, which in its natural state, is visible either to the naked eye or with the aid of an optical filter and/or applied stimulation, e.g., ultraviolet light, to promote fluorescence. Examples of colored labels which can be used include metallic sol particles, gold sol particles, dye sol particles, dyed latex particles or dyes encapsulated in liposomes. Other direct labels include radionuclides and fluorescent or luminescent moieties. Indirect labels such as enzymes can also be used according to the invention. Various enzymes are known for use as labels such as, for example, alkaline phosphatase, horseradish peroxidase, lysozyme, glucose-6-phosphate dehydrogenase, lactate dehydrogenase and urease. For a detailed discussion of enzymes in immunoassays see Engvall, Enzyme Immunoassay ELISA and EMIT, Methods of Enzymology, 70, 419-439 (1980).

The antibody can be attached to a surface. Examples of useful surfaces on which the antibody can be attached for the purposes of detecting the desired antigen include nitrocellulose, PVDF, polystyrene, and nylon. The surface or support may also be a porous support (see, e.g., U.S. Pat. No. 7,939,342). The assays can be carried out in various assay device formats including those described in U.S. Pat. Nos. 4,906,439; 5,051,237 and 5,147,609 to PB Diagnostic Systems, Inc.

In some embodiments of the processes, assays and methods described herein, detecting the level of antibodies reactive to GalNAc-T13 or a variant thereof includes contacting the sample from the cancer patient with an antibody or a fragment thereof that specifically binds GalNAc-T13 or a variant thereof, forming an antibody-protein complex between the antibody and GalNAc-T13 or a variant thereof present in the sample, washing the sample to remove the unbound antibody, adding a detection antibody that is labeled and is reactive to the antibody bound to GalNAc-T13 or a variant thereof in the sample, washing to remove the unbound labeled detection antibody and converting the label to a detectable signal, wherein the detectable signal is indicative of the level of GalNAc-T13 or a variant thereof in the sample from the patient. In some embodiments, the effector component is a detectable moiety selected from the group consisting of a fluorescent label, a radioactive compound, an enzyme, a substrate, an epitope tag, electron-dense reagent, biotin, digonigenin, hapten and a combination thereof. In some embodiments, the detection antibody is labeled by covalently linking to an enzyme, labeled with a fluorescent compound or metal, labeled with a chemiluminescent compound. The level of GalNAc-T13 may be obtained by measuring a light scattering intensity resulting from the formation of an antibody-protein complex formed by a reaction of GalNAc-T13 in the sample with the antibody, wherein the light scattering intensity of at least 10% above a control light scattering intensity indicates the likelihood of chemotherapy resistance.

In various embodiments of the processes, assays and methods of the invention, an increased likelihood of chemotherapy resistance may result in poor prognosis wherein the poor prognosis comprises decreased survival likelihood, shortened life expectancy, or enhanced tumor stemness.

In various embodiments of the processes, assays and methods of the invention, the process described herein further comprises prescribing a first therapy to the subject if the subject has a good prognosis or prescribing a second therapy, or both the first therapy and the second therapy, to the subject if the subject has a poor prognosis.

### Reference Values

In various embodiments of the processes, assays and methods described herein, the reference value is based on the expression level of GalNAc-T13 or a variant thereof. In one embodiment, the expression level is in a cancer cell. In another embodiment, the expression level is in a non-cancer cell. In an additional embodiment, the expression level is in any cell. In some embodiments, the reference value is the mean or median expression level of GalNAc-T13 or a variant thereof in a population of subjects that do not have cancer. In other embodiments, the reference value is the mean or median expression level of GalNAc-T13 or a variant thereof in a population of subjects that have cancer and respond to chemotherapy. In some embodiments the reference value that comprises the population of subjects that have cancer and respond to chemotherapy show undetectable expression of GalNAc-T13 or show reduced expression of GalNAc-T13. In additional embodiments, the reference value is the expression level of GalNAc-T13 or a variant thereof in a sample obtained from the subject from a different (for example, an earlier) time point, such as during diagnosis, before treatment, after treatment or a combination thereof. In some embodiments, the cancer is lung cancer.

In various embodiments, the expression level of GalNAc-T13 or a variant thereof in the cancer subject compared to the reference value is increased by at least or about 10%, 20%, 30%, 40%, 50%, 60%, 70%, 80%, 90% or 100%. In various embodiments, the expression level of GalNAc-T13 or a variant thereof in the cancer subject compared to the reference value is increased by at least or about 1-fold, 2-fold, 3-fold, 4-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold or a combination thereof.

### Therapies

In accordance with various embodiments of the invention, the therapies described herein may be selected, used and/or administered to treat a cancer patient (for example a lung cancer patient). In various embodiments, the first therapy may be any one or more of surgery, radiation, chemotherapy, immunotherapy, vaccine or combinations thereof. In various embodiments, second therapy is administered if GalNAc-T13 or a variant thereof is present in the subject or the levels of GalNAc-T13 or a variant thereof have increased in the subject, which is indicative of chemotherapy resistance in the cancer (for example, NSCLC) patient. Second therapy includes surgery, radiation, immunotherapy, vaccine or combinations thereof. In some embodiments, chemotherapy may be included in the second therapy with administering higher dosages of chemotherapeutic drugs, administering combinations of chemotherapeutic drugs or a combination thereof.

In some embodiments, chemotherapeutic agents may be selected from any one or more of cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5 -fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.).

In various embodiments, first and/or second therapies include use of chemotherapeutic agents to treat lung cancer. Such agents include but are not limited to Abitrexate, Abraxane (Paclitaxel Albumin-stabilized Nanoparticle Formulation), Afatinib, Alimta (Pemetrexed Disodium), Avastin (Bevacizumab), Bevacizumab, Carboplatin, Cisplatin, Crizotinib, Erlotinib Hydrochloride, Folex (Methotrexate), Folex PFS (Methotrexate), Gefitinib, Gilotrif (Afatinib), Gemcitabine Hydrochloride, Gemzar (Gemcitabine Hydrochloride), Iressa (Gefitinib), Methotrexate, Methotrexate LPF, Mexate, Mexate-AQ, Nivolumab, Necitumumab, Paclitaxel, Paclitaxel Albumin-stabilized Nanoparticle Formulation, Paraplat (Carboplatin), Paraplatin (Carboplatin), Pemetrexed Disodium, Platinol (Cisplatin), Platinol-AQ (Cisplatin), Tarceva (Erlotinib Hydrochloride), Taxol (Paclitaxel), Xalkori (Crizotinib) or a combination thereof.

In various embodiments, therapies include, for example, radiation therapy. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In various embodiments, therapies include, for example, immunotherapy. Immunotherapy may comprise, for example, use of cancer vaccines and/or sensitized antigen presenting cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines.

In various embodiments, therapies include, for example, hormonal therapy, Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

The duration and/or dose of treatment with anti-cancer therapies may vary according to the particular anti-cancer agent or combination thereof. An appropriate treatment time for a particular cancer therapeutic agent will be appreciated by the skilled artisan. The invention contemplates the continued assessment of optimal treatment schedules for each cancer therapeutic agent, where the genetic signature of the cancer of the subject as determined by the methods of the invention is a factor in determining optimal treatment doses and schedules.

In various embodiments, the subject for whom predicted efficacy of an anti-cancer therapy is determined, is a mammal (e.g., mouse, rat, primate, non-human mammal, domestic animal such as dog, cat, cow, horse), and is preferably a human. In another embodiment of the methods of the invention, the subject has not undergone chemotherapy or radiation therapy. In alternative embodiments, the subject has undergone chemotherapy or radiation therapy (e.g., such as with cisplatin, carboplatin, and/or taxane). In related embodiments, the subject has not been exposed to levels of radiation or chemotoxic agents above those encountered generally or on average by the subjects of a species. In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient, or e.g., the subject is given the anti-cancer therapy prior to removal of the cancerous tissue.

### Samples

Samples, such as cancer cells, cancerous tissue, plasma and/or blood, could be collected preferably at the time of biopsy for diagnosis of the cancer. This would allow the best chance to design a course of treatment that would best serve the patient. For example, if expression of GalNAc-T13 or a variant thereof has increased, the patient may require a more aggressive treatment course compared to another patient with a cancer that does not have increased expression of GalNAc-T13. It is also possible to obtain cancerous tissue, plasma and/or blood after cancer treatment (e.g., surgery) or during cancer treatment (e.g., radiation, chemotherapy etc.). This would allow for a change in treatment course or decision on the course of treatment with the prospect of recurrence. In various embodiments, the cancer is a lung cancer. In some embodiments, the lung cancer is a non-small cell lung cancer. In an embodiment, the NSCLC is an adenocarcinoma.

The steps involved in the current invention comprise obtaining either through surgical biopsy or surgical resection, a sample of the patient's lung tumor and matching blood sample from the patient. Alternatively, a sample can be obtained through primary patient harvested lung tumor stem cells, primary patient lung tumor derived cell lines, or archived patient samples in the form of FFPE (Formalin fixed, paraffin embedded) samples, or fresh frozen lung tumor samples. This invention also allows for the possibility of retrospectively evaluating the above mentioned parts of this invention (i.e. likelihood of survival, estimated life expectancy and the potential of acquiring this mutation in the future).

Patient's tumor sample is then used to extract Deoxyribonucleic acid (DNA) using the standard protocol designated “QIAamp DNA Mini and Blood Mini kit” or for FFPE samples “QIAamp DNA FFPE Tissue kit” commercially available from Qiagen®. The above and following procedures require informed consent from patients.

The invention provides a system for determining responsiveness of a cancer cell to chemotherapy wherein the cancer cell is obtained from a cancer patient. The system includes a sample analyzer configured to produce a signal for mRNA encoding GalNAc-T13 present in the cancer cell obtained from the cancer patient and a computer sub-system programmed to calculate, based on the mRNA whether the signal is greater than or not greater than a reference value.

The invention also provides a system for determining responsiveness of a cancer cell to chemotherapy wherein the cancer cell is obtained from a cancer patient. The system comprises a sample analyzer configured to produce a signal when a GalNAc-T13-specific antibody binds GalNAc-T13 in the cancer cell obtained from a cancer patient and a computer sub-system programmed to calculate, based on the antibody binding whether the signal is greater than or not greater than a reference value.

In some embodiments, the computer sub-system is programmed to compare the mRNA to determine a likelihood of responsiveness of said cancer cell to chemotherapy based on an algorithm that classifies the patient as likely to responds to a chemotherapy-comprising therapy if GalNAc-T13 expression is increased and as unlikely to respond to chemotherapy-comprising therapy if the GalNAc-T13 is not increased.

The invention further provides a computer program product embodied in a computer readable medium that, when executed on a computer, performs steps comprising detecting GalNAc-T13 expression in a sample comprising a cancer cell obtained from a cancer patient and comparing the GalNAc-T13 expression to a reference value. A diagnostic kit for detecting a likelihood of a cancer patient responding to chemotherapy comprising no more than 10 probes comprising a combination of detectable labeled probes or primers for GalNAc-T13 and a computer program product described herein.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

O-gylcosylation alterations occur in most carcinomas, resulting in the expression of molecules which may constitute useful targets for diagnostic and therapy. GalNAc-T13 enzyme catalyzes a key step in the initiation of O-glycosylation. It is overexpressed in metastatic neuroblastoma, and has been correlated with the prognosis of patients with this tumor. In resected lung cancer specimens there is no information about GalNAc-T13 expression.

As detailed below, Applicants used tumor tissue microarrays containing 443 NSCLCs, including 249 adenocarcinomas (ADCA) and 122 squamous cell carcinomas (SCC). Immunohistochemistry was performed using a monoclonal antibody specific against GalNAc-T13. The cytoplasmic expression of the enzyme was quantified using a four-value intensity score (0, 1+, 2+, and 3+) and the percentage (0-100%) of the extent of reactivity in each tissue core. The final score was then obtained by multiplying the intensity and reactivity extension values (range, 0-300). The patients were divided into 2 groups: with (n=72, WNA) and without neoadjuvant (n=371, WONA) chemotherapy.

As described below, Applicants found frequent GalNAc-T13 expression in NSCLC, without significant differences between WNA and WONA (p=0.20) groups. ADCAs expressed higher levels of the enzyme than SCCs in both groups (WNA, p=0.02; and, WONA, p<0.0001). In the ADCA patients, with or without neoadyuvant, GalNAc-T13 expression is different according histology pattern (WNA: p=0.002 and WONA: p=0.044) showed higher value with the presence of solid histology pattern and lower in lepidic histology pattern. Using Spearman Correlation test, GalNAc-T13 correlated significantly with EpCAM (p<0.001) and TTF-1 (p<0.01) expression, In ADCAs, we found no correlation between GalNAc-T13 expression and EGFR and KRAS mutation status and the presence of EML4-ALK fusion gene. In the ADCA-WNA subset, high GalNAc-T13 expression level was associated with worse OS (p<0.01, HR=5.2), not significative in RFS (p—0.15, HR=1.8). In contrast, association between GalNAc-T13 expression and outcome in the ADCAWONA subset of patients was not found. GalNAc-T13 is frequently expressed in NSCLC and associates with poor prognosis in patients with ADCA who received neoadjuvant chemotherapy. Data herein suggests that GalNAc-T13 is a novel marker associated with chemoresistance in NSCLC.

### Example 1

### Experimental Methods

### Cell Lines

Human lung cancer cell lines representing different histological types, stages and conditions of disease (SK-MES-1, A549, NCI-H1703, NCI-H838, NCI-H1755, NCI-H526, NCI-H1650, NCI-H1975, H69AR and NL-20) were purchased from ATCC and in vitro cultured according with provider's instructions.

Production of anti-GalNAc-T13 Monoclonal Antibody (T13.5 Hybridoma Production Protocol)

### Immunization

A synthetic peptide of GalNAc-T13 was selected in the region which displays very high variability among GalNAc-Ts family members (RSLLPALRAVISRNQE, accession number BAC54545) (Biosynthesis). Four Balb/c female mice of 8 weeks of age from the Division of Veterinary Laboratories (DI.LA.VE., Montevideo, Uruguay) were used.

Mice were immunized three times at 2 week intervals (d0, i.p. and d14, d21, s.c.). The immunization mixture contained 50% v/v 100 μg of the synthetic peptide carried by KLH in PBS and Freund's adjuvant (complete for the first immunization and incomplete for the followings) in a total volume of 100 μl.

Mice were bled before the first immunization and at d24 and d31 for serum collection (˜100 μl). Serum antibody titer was determined by ELISA after d31 sampling. The titer reached 1/3000. The chosen mouse was boosted s.c. with a similar mixture (100 μg of the KLH-synthetic peptide in PBS and incomplete Freund's adjuvant) three days before fusion.

### Fusion Protocol

### Myeloma Cells

SP2/O myeloma cell line was thawed 10 days before fusion and cultured in DMEM 2 mM glutamine, 1 mM sodium pyruvate, 10% SBF at 37° C. in a 5% CO₂ humidified atmosphere. The day before fusion, myeloma cells were split into fresh bottles with culture medium supplemented with 20% SBF. All mediums (DMEM supplemented with 2 mM glutamine and 1 mM sodium pyruvate, with and without SBF 20%) and PEG 1,450 (Sigma) were pre-warmed to 37° C. before use. Myeloma cells were pooled and counted, then left in a 50 ml tube in complete DMEM without SBF in incubator during spleen cell recovery.

### Spleen Cells

The mouse was euthanized via cervical dislocation and placed in a beaker containing 70% ethanol. Spleen was removed in a laminar flow hood using aseptic techniques, and transferred to a Potter-Elvchjem (Sigma) containing 3 ml of complete DMEM without scrum. Spleen was homogenized and splenocytes were transferred to a 50 ml tube in complete DMEM without SBF and counted. Splenocytes and myeloma cells were centrifuged at 1000 rpm for 5 min, and then resuspended in 10 ml of complete medium without SBF. Myeloma cells and splenocytes were pooled in a freshly 50 ml tube at a ½ proportion, and centrifuged in the same conditions.

### Fusion

Supernatant was poured off from the cell mixture and the pellet was gently resuspended, by finger-flicking, in the remaining liquid. 1.5 ml of 37° C. pre-warmed PEG were slowly added by 1 min 30 seconds through gently rotation of tube, and then 20 ml of medium without serum was added slowly by 3 min. Cells were centrifuged at 1000 rpm for 5 min and plated in HAT medium 20% SBF in 96 well culture-plates (200 μl/well), then placed in the incubator at 37° C. in a 5% CO₂ humidified atmosphere. Medium was 50% replaced by freshly pre-warmed HAT medium at days 4 and 7.

### Clone Testing

After 10-14 days, when clones were visible by naked eye, plates were screened by inverted microscope in order to choose wells to be tested. Screening was performed by ELISA with microtiter plates coated with the specific peptide carrying by BSA. Cells from ELISA positive wells were transferred into 24 well culture plates, counted, and cloned by limit dilution into 96 well culture plates containing a feeder layer prepared with Balb/c splenocytes on the eve. 10-15 days later all wells having a unique clone were retested by ELISA and positive clones were expanded in 24 well plates, then 25 cm² bottles and stored in SBF 10% DMSO in liquid nitrogen.

### Analysis of Monoclonal Antibody Specificity by Surface Plasmon Resonance

Interactions between the mAb 13.5 and synthetic peptides were analyzed by performing surface plasmon resonance experiments on a BIAcore 3000 instrument (GE Healthcare, Sweden). Purified mAb was coupled to an activated carboxymethylated dextran CM-5 sensor surfaces (SA sensorchip, GE Healthcare, Sweden). The peptides were diluted in HBS-EP buffer (10 mM HEPES, 150 mM NaCl, 3 mM EDTA, and 0.005% Surfactant P20, pH 7.4) and were passed over the sensorchip. All experiments were run in duplicates at a 30 μL/min flow rate, a contact time of 180 s and a dissociation time of 360 s, with the biosensor instrument thermostated at 25° C. After dissociation the sensor chip was regenerated by injecting 10 mM glycine—HCl (pH 2.5) at the end of each experiment. All data processing was carried out using the BIAevaluation 4.1 software provided by BIAcore.

### RT-PCR

Total RNA was extracted from lung cancer cell lines with Tri-Reagent (Sigma) according to the manufacturer's instructions. Two μg of total RNA were included for first strand cDNA synthesis by using 200 units of M-MLV reverse transcriptase (Amersham, Piscataway, N.J.) in the presence of 2 μl 10 mM of each deoxynucleotide triphosphate (dNTPs) and 200 ng of random hexamers (Fermentas Inc, Maryland) in a 241 total reaction volume. After incubation at 37° C. for 1 hr, the mixture was heated to 70° C., snap-cooled and stored at −20° C. Amplification of a 425 by of GALNT13 transcripts was performed using the follow specific primers: 5′-ACATCTATCCGGACTCCC-3′ and 5′-TCATGTGCCCAAGGTCATGTTCC-3′ (accession number AJ505991). The PCR mixture (total reaction volume of 25 μl) includes 20 mM Tris-HCl (pH 8.4), 50 mM KCl, 2.5 mM MgCl₂, 200 μM dNTPs, 300 nM each primer and 1 unit of Taq DNA polymerase (Fermentas Inc, Maryland). Amplification was performed for 35 cycles under the following conditions: 45 sec at 95° C., 1 min at 62° C. and 1 min at 72° C. PCR products (15 μl) were analyzed by electrophoresis on 2% agarose gels by direct visualization after ethidium bromide staining.

### Immunofluorescence Microscopy

Cells plated on glass coverslips were washed with PBS, fixed in methanol-acetone 50% for 10 min and stored a −20° C. until use. Coverslips were then defrosted, rehydrated in PBS, and blocked in 30% goat serum for 20 min. Primary antibody T13.5 was then incubated for 1 hr at room temperature and after three washes for 5 min each in PBS, secondary antibody conjugated with Alexa Fluor® 488 Dye was incubated for 1 hour at room temperature. Monolayers were counterstained with DAPI, mounted in PBS-glycerol 50% and analyzed by regular epifluorescence microscopy or by confocal immunofluorescence microscopy using a Zeiss LSM 510 confocal microscope.

### Patients and Immunohistochemical Analysis

We collected tumor tissue from surgically resected primary lung adenocarcinomas from patients that had undergone surgical resection with curative intent, between the years 2003 to 2005, at the University of Texas MD Anderson Cancer Center, Houston, Tex. Clinicopathologic information was retrieved from the electronic clinical records in all cases and included age, sex, smoking history and status (current, former, or never), tumor size, tumor stage according to the International Association for the Study of Lung Cancer (IASLC) [Detterbeck, 2009 classification systems], neoadjuvant and adjuvant treatment, and follow-up information for RFS and OS rates.

Tissue microarrays (TMA) were constructed with paraffin embedded formalin fixed tissues from 443 NSCLC patients surgically resected. We performed immunohistochemistry using a monoclonal antibody specific for ppGalNAc-T13 (mAb T13.5) on 5-uM-thick TMAs sections. Tissue sections were deparaffinized and hydrated, and antigen retrieval was performed in pH 6.0 citrate buffer in a decloaking chamber (121° C.×30 minutes, 90° C.×10 minutes) and washed with Tris buffer. Peroxidase blocking was performed at room temperature for 15 minutes with 3% H₂O₂ in methanol. Protein blocking was performed with Dako serum-free protein block for 30 minutes. The slides were incubated with primary antibody at room temperature for 90 minutes and washed with Tris buffer, followed by incubation with Envision Dual-Link system-horseradish peroxidase (Dako) for 30 minutes.

Staining was developed with 0.5% 3,3′-diaminobenzidine, freshly prepared with imidazoleHCl buffer, pH 7.5, containing hydrogen peroxide and an antimicrobial agent (Dako) for 5 minutes and then counterstained with hematoxylin, dehydrated, and mounted.

The cytoplasm immunostainings for GalNAc-T13 was quantified using a four-value intensity score (0, 1+, 2+, and 3+) and the percentage (0-100%) of the extent of reactivity in each core. The final score was then obtained by multiplying the intensity and reactivity extension values (range, 0-300) quantify. According the distribution in our population, was considered the median as cut-off value: 40. The population was divided into 2 groups, as they had received or not neoadjuvant therapy, 72 patients with neoadjuvant (WNA) and 371 patients without neoadjuvant (WONA), then we analyzed adenocarcinoma (ADCA) and squamous (SQM) as independent groups.

### Example 2

### Generation of a Monoclonal Antibody Specific for GalNAc-T13 Useful for Immunohistochemical Studies in Paraffin Embedded Tissues.

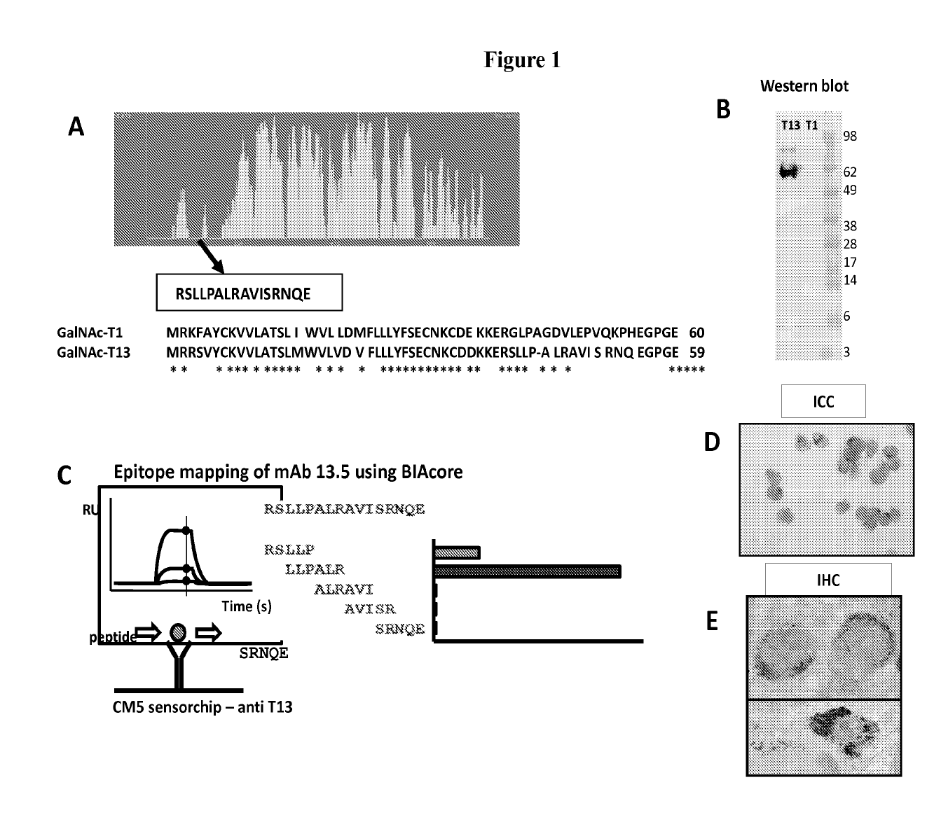

Considering that GalNAc-T13 displays 84% homology compared with GalNAc-T1 we immunized mice with a KLH-conjugated specific motif (RSLLPALRAVISRNQE) of GalNAc-T13, without any homology with GalNAc-T1 sequence (FIG. 1A). Selection of specific hybridomas was performed by ELISA, screening against BSA-conjugated GalNAc-T13 peptide. One of the mAbs, T13.5, strongly reactive against the synthetic peptide, was used for further characterization. We evaluated the mAb T13.5 reactivity in Western blot using GalNAc-T1 and -T13 expressed in baculovirus. We found that the antibody reacts with GalNAc-T13 but not with GalNAc-T1 (FIG. 1B), confirming the specificity of this antibody for GalNAc-T13 and that it binds to denatured forms of the protein. To determine which amino acid residues are crucial for mAb T13.5 binding we mapped the epitope using overlapping peptides covering the sequence RSLLPALRAVISRNQE. Peptide binding to immobilized antibody was assessed using BIAcore (FIG. 1C). The results obtained indicate that the epitope of mAb T13.5 could be mapped to residues LLPLAR. Considering that ppGalNAc-T13 expression was previously reported in neuroblastoma (Berois et al., 2006a), we performed an immunocytochemical analysis using MAb T13.5 on the IMR-32 cell line (FIG. 1D). We found a strong staining preponderantly detected in the perinuclear region, as expected for a glycosyltransferase localized in Golgi apparatus. The immunohistochemical evaluation in neuroblastoma tumors (FIG. 1E) demonstrates that mAb T13.5 is able to detect GalNAc-T13 in paraffin embedded tissues used in the pathological routine diagnostic.

### Example 3

### GalNAc-T13 is Expressed in Human Lung Cancer Cells.

Seventy two lung cancer patients presenting locally advanced disease received neo-adjuvant therapy prior surgery. Patients were treated with one of carboplatin and paclitaxel, or carboplatin and docetaxel, or carboplatin and gemcitabine, or carboplatin and pemetrexed, or carboplatin and etoposid, or cisplatin and docetaxel, or cisplatin and etoposide, or cisplatin and gemcitabine, or paclitaxel and cetuximab.

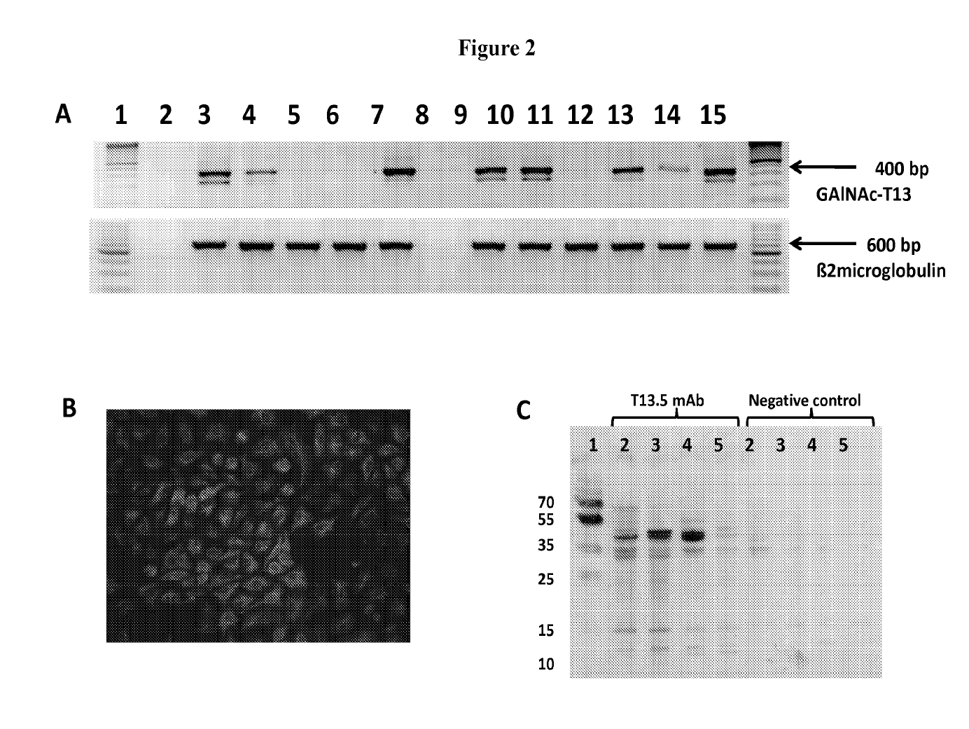

It was reported that GalNAc-T13 is a glycosyltransferase specifically expressed in neuronal tissue (Zhang et al., 2003). Here, evaluating a panel of human lung cancer cell lines by RT-PCR, we found the mRNA coding GalNAc-T13 in A549, NCI-H1703, NCI-H1755, NCI-H526, NCI-H1650, H69AR and NL-20 cell lines (FIG. 2A). In contrast, the RT-PCR analysis was negative in SK-MES-1, NCI-H838 and NCI-H1975 cell lines. We confirm at protein level the expression of GalNAc-T13 in human lung cancer cells using immunofluorescence microscopy (FIG. 2B) and Western blot (FIG. 2C). The results obtained by RT-PCR (FIG. 2A) and Western blot (FIG. 2C) suggest that splice variants of GalNAc-T13 are expressed in human lung cancer.

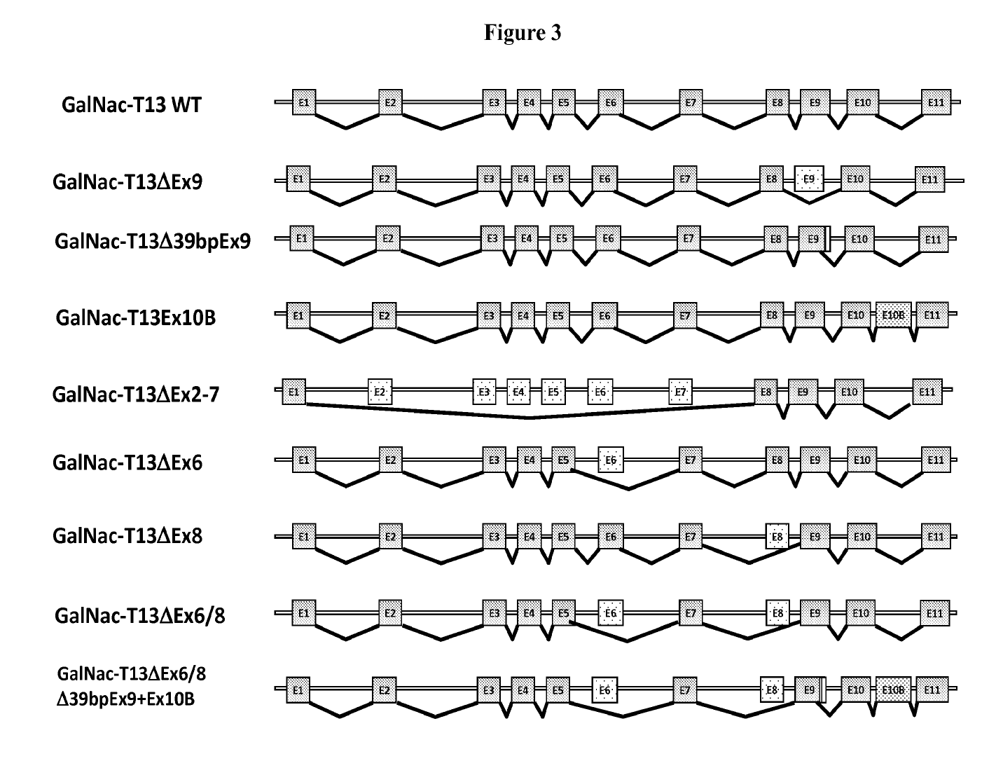

Using a strategy based in colony-PCR and nucleotide sequencing we demonstrate, for the first time, a large family of splice variants of GalNAc-T13 (FIG. 3). GalNAc-T13 wild type is encoded by the sequences set forth in SEQ ID NOs: 1 and 2. The splice variant GalNAc-T13ΔEx9 having a deletion of exon 9 of GalNAc-T13 is encoded by the sequence set forth in SEQ ID Nos: 3 and 4. The splice variant GalNAc-T13Δ39bpEx9 having a deletion of 39 nucleotides in exon 9 of GalNAc-T13 is encoded by the sequence set forth in SEQ ID Nos: 5 and 6. The splice variant GalNAc-T13ΔEx10B having a deletion of exon 10B of GalNAc-T13 is encoded by the sequence set forth in SEQ ID Nos: 7 and 8. The splice variant GalNAc-T13ΔEx2-7 having a deletion of exons 2-7 of GalNAc-T13 is encoded by the sequence set forth in SEQ ID Nos: 9 and 10. The splice variant GalNAc-T13ΔEx6 having a deletion of exon 6 of GalNAc-T13 is encoded by the sequence set forth in SEQ ID Nos: 11 and 12. The splice variant GalNAc-T13ΔEx8 having a deletion of exon 8 of GalNAc-T13 is encoded by the sequence set forth in SEQ ID Nos: 13 and 14. The splice variant GalNAc-T13ΔEx6ΔEx8 having a deletion of exons 6 and 8 of GalNAc-T13 is encoded by the sequence set forth in SEQ ID Nos: 15 and 16. The splice variant GalNAc-T13ΔEx6ΔEx8Δ39bpEx9ΔEx10B having a deletion of exon 6, exon 8, 39 nucleotides of exon 9 and exon 10B of GalNAc-T13 is encoded by the sequence set forth in SEQ ID Nos: 17 and 18.

### Example 4

### GalNAc-T13 as a Novel Immunohistochemical Marker Associated to Chemoresistance in NSCLC.

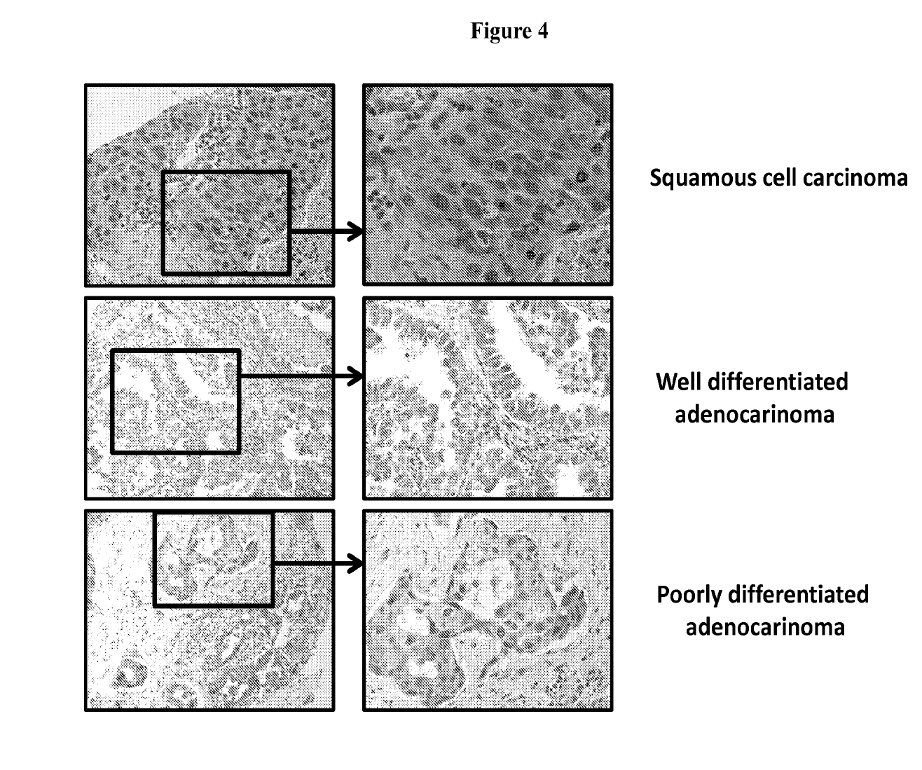

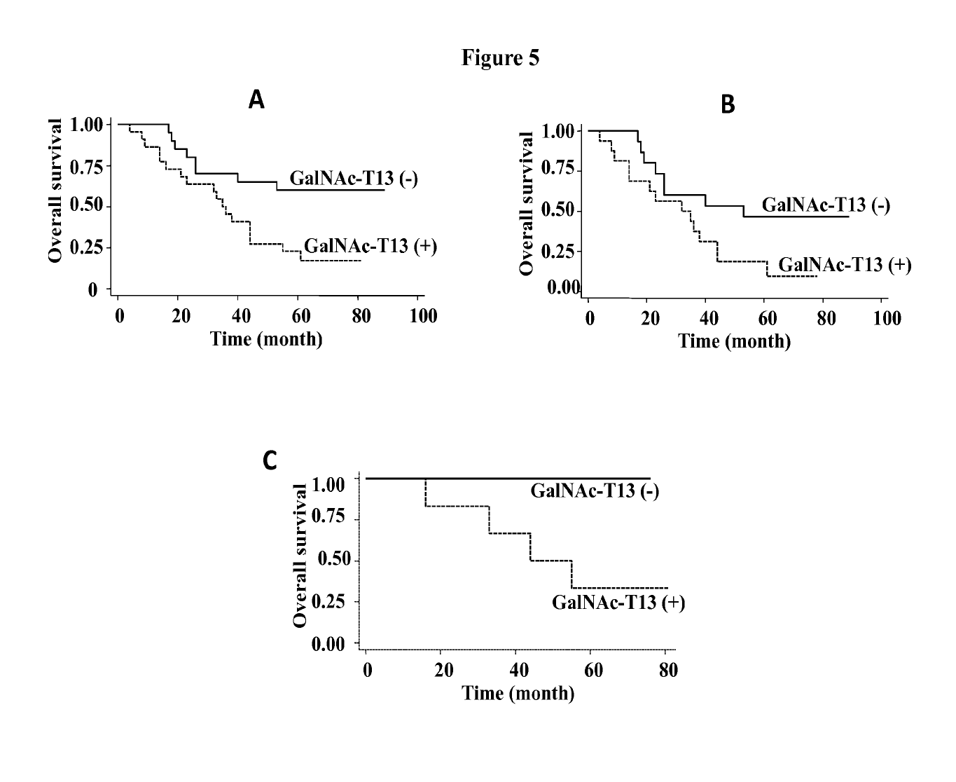

MAb T13.5 immunostaining was evaluated in 443 primary tumors from lung cancer patients. The characteristics of patients and tumors are indicated in the Table I. mAb T13.5 always showed a diffuse cytoplasmatic staining pattern (FIG. 4). We found that GalNAc-T13 expression was significantly higher in adenocarcinomas than in squamous cell tumors (p<0.001). Non correlation was found between GalNAc-T13 expression and EGFR and KRAS mutation status and the presence of EML4-ALK fusion gene. In patients with adenocarcinomas receiving neoadjuvant treatment, high GalNAc-T13 expression level was associated with worse overall survival (p<0.01, HR=5.2) (FIG. 5A). Similar results were observed in patients with advanced tumors (FIG. 5B) as well as in early stage adenocarcinoma patients (FIG. 5C). In contrast, we did not find any association between GalNAc-T13 expression and outcome in adenocarcinoma patients without neoadjuvant treatment. These data strongly suggest that GalNAc-T13 is a novel marker associated to chemoresistance in lung adenocarcinoma patients.

TABLE 1
Characteristics of the study population according with GalNAcT13
expression in lung cancer primary tumor

| | T13-H¹ | | T13-L² | | Total | |
|---|---|---|---|---|---|---|
| | n (%) n | | (%) | | n (%) | |
| Gender | | | | | | |
| Male | 107 | (49) | 111 | (51) | 218 | (100) |
| Female | 97 | (43.1) | 128 | (56.9) | 225 | (100) |
| Age | | | | | | |
| (median 66 ± 10.3 years) | | | | | | |
| <66 | 95 | (48) | 103 | (52) | 198 | (100) |
| ≧66 | 109 | (44.5) | 136 | (55.5) | 245 | (100) |
| Tobacco Current | 86 | (45.5) | 103 | (54.5) | 189 | (100) |
| Former Never | 97 | (47.5) | 107 | (52.5) | 204 | (100) |
| | 21 | (42) | 29 | (58) | 50 | (100) |
| Stage | | | | | | |
| I | 106 | (47) | 120 | (53) | 226 | (100) |
| II | 43 | (39.8) | 65 | (60.2) | 108 | (100) |
| III | 46 | (47.9) | 50 | (52.1) | 96 | (100) |
| IV | 9 | (69.2) | 4 | (30.8) | 13 | (100) |
| Histopathology | | | | | | |
| Adenocarcinoma | 160 | (54.2) | 135 | (45.8) | 295 | (100) |
| acinar | 30 | (51.7) | 28 | (48.3) | 58 | (100) |
| solid | 76 | (64.4) | 42 | (35.6) | 118 | (100) |
| papilar | 19 | (47.5) | 21 | (52.5) | 40 | (100) |
| lepidic | 27 | (39.7) | 41 | (60.3) | 68 | (100) |
| NA | 8 | (72.7) | 3 | (27.3) | 11 | (100) |
| Squamous cell carcinoma | 44 | (29.7) | 104 | (70.3) | 148 | (100) |
| Grade | | | | | | |
| Well differentiated | 16 | (32) | 34 | (68) | 50 | (100) |
| Moderately differentiated | 101 | (43.3) | 132 | (56.7) | 233 | (100) |
| Poorly differentiated | 82 | (53.9) | 70 | (46.1) | 152 | (100) |
| NA | 5 | (62.5) | 3 | (37.5) | 8 | (100) |

¹GalNAcT13 high expression
²GalNAcT13 low or non-expression

### REFERENCES

1. Adamczyk B, Tharmalingam T, Rudd P M. Glycans as cancer biomarkers. Biochim Biophys Acta. 2012; 1820:1347-53.
2. Altekruse S F, Kosary C L, Krapcho M. SEER Cancer Statistics Review, 1975-2007. National Cancer Institute; 2010. Available at: http://seer.cancer.gov/csr/1975_2007/.
3. Azzoli C G, Baker S Jr, Temin S, Pao W, Aliff T, Brahmer J, Johnson D H, Laskin J L, Masters G, Milton D, Nordquist L, Pfister D G, Piantadosi S, Schiller J H, Smith R, Smith T J, Strawn J R, Trent D, Giaccone G; American Society of Clinical Oncology. Clinical Practice Guideline update on chemotherapy for stage IV non-small-cell lung cancer. J. Clin. Oncol. 2009; 27:6251-6266.
4. Beasley M B, Brambilla E, Travis W D. The 2004 World Health Organization classification of lung tumors. Semin Roentgenol 2005; 40:90-7.
5. Bennett E P, Mandel U, Clausen H, Gerken T A, Fritz T A, Tabak L A. Control of Mucin-Type O-Glycosylation—A Classification of the Polypeptide GalNAc-transferase Gene Family. Glycobiology. 2012; 22:736-56.
6. Berois N, Blanc E, Ripoche H, Mergui X, Trajtenberg F, Cantais S, Barrois M, Dessen

P, Kågedal B, Bénard J, Osinaga E, Raguénez G. ppGalNAc-T13: a new molecular marker of bone marrow involvement in neuroblastoma. Clin Chem 2006; 52:1701-12.
7. Berois N, Mazal D, Ubillos L, Trajtenberg F, Nicolas A, Sastre-Garau X Magdelenat H, Osinaga E. UDP-N-Acetyl-D-Galactosamine: Polypeptide N-Acetylgalactosaminyltransferase-6 as a new immunohistochemical breast cancer marker. J Histochem Cytochem 2006;54:317-28.
8. Freire T, Berois N, Sóñora C, Varangot M, Barrios E, Osinaga E. UDP-N-acetyl-D-galactosamine:polypeptide N-acetylgalactosaminyltransferase 6 (ppGalNAc-T6) mRNA as a potential new marker for detection of bone marrow-disseminated breast cancer cells. Int J Cancer 2006; 119:1383-8.
9. Gu C, Oyama T, Osaki T, Li J, Takenoyama M, Izumi H, Sugio K, Kohno K, Yasumoto K. Low expression of polypeptide GalNAc N-acetylgalactosaminyl transferase-3 in lung adenocarcinoma: impact on poor prognosis and early recurrence. Br J Cancer. 2004; 90:436-42.
10. Guda K, Moinova H, He J, Jamison O, Ravi L, Natale L, Lutterbaugh J, Lawrence E, Lewis S, Willson J K, Lowe J B, Wiesner G L, Parmigiani G, Barnholtz-Sloan J, Dawson D W, Velculescu V E, Kinzler K W, Papadopoulos N, Vogelstein B, Willis J, Gerken T A, Markowitz S D. Inactivating germ-line and somatic mutations in polypeptide N-acetylgalactosaminyltransferase 12 in human colon cancers. Proc Natl Acad Sci USA. 2009; 106:12921-5.
11. Jemal A, Siegel R, Xu J, Ward E. Cancer Statistics, 2010. CA Cancer J Clin 2010; 60:277- 300.
12. Lynch T J, Bell D W, Sordella R, Gurubhagavatula S, Okimoto R A, Brannigan B W, Harris P L, Haserlat S M, Supko J G, Haluska F G, Louis D N, Christiani D C, Settleman J, Haber D A. Activating mutations in the epidermal growth factor receptor underlying responsiveness of non-small-cell lung cancer to gefitinib. N. Engl. J. Med. 2004; 350:2129-2139.
13. Mandel U, Hassan H, Therkildsen M H, Rygaard J, Jakobsen M H, Juhl B R, Dabelsteen E, Clausen H. Expression of polypeptide GalNAc-transferases in stratified epithelia and squamous cell carcinomas: immunohistological evaluation using monoclonal antibodies to three members of the GalNAc-transferase family. Glycobiology 1999; 9:43-52.
14. Mok T S. Personalized medicine in lung cáncer: what we need to know. Nat Rev Clin Oncol. 2011; 8:661-8.
15. Miyahara N, Shoda J, Kawamoto T, Furukawa M, Ueda T, Todoroki T, Tanaka N, Matsuo K, Yamada Y, Kohno K, lrimura T. Expression of UDP-N-acetyl-alpha-D-galactosaminepolypeptide N-acetylgalactosaminyltransferase isozyme 3 in the subserosal layer correlates with postsurgical survival of pathological tumor stage 2 carcinoma of the gallbladder. Clin Cancer Res 2004; 10:2090-9.
16. Paez J G, Jänne P A, Lee J C, Tracy S, Greulich H, Gabriel S, Herman P, Kaye F J, Lindeman N, Boggon T J, Naoki K, Sasaki H, Fujii Y, Eck M J, Sellers W R, Johnson B E, Meyerson M. EGFR mutations in lung cancer: correlation with clinical response to gefitinib therapy. Science 2004; 304:1497-1500.
17. Reis C A, Osorio H, Silva L, Gomes C, David L. Alterations in glycosylation as biomarkers for cancer detection. J Clin Pathol. 2010; 63:322-9.
18. Saijo N. Critical comments for roles of biomarkers in the diagnosis and treatment of cancer. Cancer Treatment Reviews 2012; 38:63-67.
19. Taniuchi K, Cerny R L, Tanouchi A, Kohno K, Kotani N, Honke K, Saibara T, Hollingsworth M A. Overexpression of GalNAc-transferase GalNAc-T3 promotes pancreatic cancer cell growth. Oncogene. 2011; 30:4843-54.
20. Tarp M A and Clausen H. Mucin-type O-glycosylation and its potential use in drug and vaccine development. Biochim. Biophys. Acta 2008, 1780:546-563.
21. Ten Hagen K, Fritz T, Tabak, L. All in the family: the UDP-GalNAc:polypeptide N-acetylgalactosaminyltransferases. Glycobiology 2003; 13:1R-16R.
22. Zhang Y, Iwasaki H, Wang H, Kudo T, Kalka T B, Hennet T, et al. Cloning and characterization of a new human UDP-N-acetyl-alpha-D-galactosamine:polypeptide N-acetylgalactosaminyltransferase, designated pp-GalNAc-T13, that is specifically expressed in neurons and synthesizes GalNAc alpha-serine/threonine antigen. J Biol Chem 2003; 278:573-84.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application arc described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1-61. (canceled)

62. An assay for determining an increased likelihood of chemotherapy resistance in a subject in need thereof comprising:
(i) providing a biological sample from a human subject having lung cancer;
(ii) providing an antibody that specifically binds to GalNAc-T13 or a variant thereof;
(iii) contacting the biological sample with the antibody; and
(iv) detecting, using immunoassay, the level of antibody binding to GalNAc-T13 or a variant thereof, wherein the presence of binding in the biological sample from the subject relative to a reference sample is indicative of increased likelihood of chemotherapy resistance in the subject.

63. An assay for selecting a therapy for a subject having lung cancer, and optionally administering the therapy, the assay comprising:
(i) providing a biological sample from a human subject having lung cancer;
(ii) providing an antibody that specifically binds to GalNAc-T13 or a variant thereof;
(iii) contacting the biological sample with the antibody;
(iv) detecting, using immunoassay, the level of antibody binding to GalNAc-T13 or a variant thereof, wherein an increase in binding in the biological sample from the subject relative to a reference sample is indicative of increased expression of ppGalNAc and increased likelihood of chemotherapy resistance in the subject; and
(v) selecting a therapy comprising prescribing a first therapy to the subject if the subject has decreased likelihood of chemotherapy resistance or prescribing a second therapy to the subject if the subject has increased likelihood of chemotherapy resistance.

64. The assay of claim 62 or 63, wherein the antibody is a monoclonal antibody.

65. The assay of claim 64, wherein the monoclonal antibody binds the epitope LLPALR of GalNAc-T13 or a variant thereof.

66. The assay of claim 62 or 63, wherein the subject has undergone neoadjuvant chemotherapy.

67. The assay of claim 62 or 63, wherein the lung cancer is non-small cell lung cancer (NSCLC).

68. The assay of claim 67, wherein the NSCLC is adenocarcinoma.

69. The assay of claim 62 or 63, wherein the sample is tissue, blood, plasma or a combination thereof and the sample is obtained before, during or after cancer treatment.

70. The assay of claim 62 or 63, wherein the reference value is any one or more of:
(i) mean or median expression level of GalNAc-T13 or a variant thereof in a population of subjects that do not have cancer;
(ii) mean or median expression level of GalNAc-T13 or a variant thereof in a population of subjects that have cancer and respond to chemotherapy; and
(iii) expression level of GalNAc-T13 or a variant thereof from the subject in a sample obtained from a different time point.

71. The assay of claim 62, further comprising prescribing a first therapy to the subject if the subject has decreased likelihood of chemotherapy resistance or prescribing a second therapy to the subject if the subject has increased likelihood of chemotherapy resistance.

72. The assay of claim 63 or 71, wherein (i) the first therapy is any one or more of surgery, radiation, chemotherapy, immunotherapy, vaccine, or a combination thereof; and
(ii) the second therapy is any one or more of surgery, radiation, immunotherapy, vaccine, or a combination thereof; or
the second therapy is a any one or more of surgery, radiation, chemotherapy, immunotherapy, vaccine, or a combination thereof, wherein chemotherapy comprises administering to the subject one or more chemotherapeutic agents that have not been used previously to treat the subject or administering a chemotherapeutic agent previously administered to the subject at a dose higher than previously administered.
